(19) United States
(12) Patent Application Publication
(10) Pub. No.: US 2012/0095488 A1
(43) Pub. Date: Apr. 19, 2012

(54) LANCET PRICKING DEVICE

(76) Inventors: Hideaki SAEKI, Maniwa-shi (JP); Hirokazu Imori, Maniwa-shi (JP); Teruyuki Abe, Shinagawa-ku (JP)

(21) Appl. No.: 13/271,642

(22) Filed: Oct. 12, 2011

(30) Foreign Application Priority Data
Oct. 15, 2010 (JP) 2010-232659

(51) Int. Cl. A61B 17/34

(52) U.S. Cl. 606/182

(57) ABSTRACT

There is provided a lancet pricking device which is prevented from being re-used, and also the pricking pathway of the needle thereof is improved. The lancet pricking device comprising a lancet equipped with a pricking needle and a trigger for launching the pricking needle. The trigger comprises a first trigger part and a second trigger part disposed such as to cover the first trigger part. The lancet is in engagement with the first trigger part before the pricking needle is launched. When the second trigger part is pushed from the outside for the launching of the pricking needle, the displacement of the first trigger part is caused by the second trigger part, and thereby ceasing the engagement between the lancet and the first trigger part to launch the pricking needle.

### TECHNICAL FIELD

The present invention relates to a pricking device. More specifically, the present invention relates to a lancet pricking device which is used for taking a sample of blood.

### BACKGROUND ART

In order to measure a blood sugar level of a patient with diabetes, it is required to take a sample of the blood from the patient. The small amount of blood taken may be enough. Thus, a pricking device capable of taking a small amount of blood is used to measure the blood sugar level. The pricking device is generally composed of a lancet (see, for example, U.S. Pat. No. 5,385,571) and an injector. The lancet has a pricking needle capable of puncturing a predetermined region of the patient's body. The injector has a function of launching the lancet toward a predetermined region. The pricking device is set up for use by loading the lancet into the injector. Then, the lancet is launched toward the predetermined region by use of a plunger of the injector, whereby the predetermined region is pricked.

The pricking device used for taking blood from the patient with diabetes is required to be desirable in terms of hygiene and safety. In this regard, a particular attention must be paid to the handling of the used lancet. As for the used lancet, the tip of the pricking needle is exposed on a lancet body, and there may be the patient's blood adhered to the pricking needle due to the pricking. If the body of a person other than the subject of the blood sampling (for example, a nurse or medical practitioner who collects the blood sample) accidentally should touch the tip of the pricking needle, the body of such person may be pricked by the pricking needle. This will result in a wound of the body through which the patient's blood may enter the body (i.e. the body of the nurse or medical practitioner), and thus posing a risk of the infection disease.

The pricking device is required to be desirable in terms of operability in use.

### PRIOR ART DOCUMENTS

### Patent Documents

Patent Document 1: U.S. Pat. No. 5,385,571

### DISCLOSURE OF THE INVENTION

### Problems to be Solved by the Invention

The present applicant has invented the following pricking device, and filed the application regarding such device (WO 2007/018215 A1, filed date: 8 Aug. 2006, title of the invention: “PRICKING DEVICE, AS WELL AS LANCET ASSEMBLY AND INJECTOR ASSEMBLY THAT CONSTITUTE THE PRICKING DEVICE”). Referring to the accompanying drawings, the lancet assembly and the injector assembly invented by the applicant will be briefly described below (note that the term “injector assembly” will be hereinafter referred to also as “injector”). FIG. 23 shows an external appearance of a lancet assembly 100′, and FIG. 24 shows an external appearance of an injector 200′. As shown in FIG. 23, the lancet assembly 100′ composed of a lancet 101′ and a protective cover 102′. As shown in FIGS. 25 and 26, the lancet 101′ comprises a lancet body 104′, a lancet cap 106′ and a pricking needle 105′. The pricking needle 105′ made of metal is disposed in both of the lancet body 104′ and the lancet cap 106′ both of which are made of resin. The tip of the pricking needle 105′ is covered with the lancet cap 106′, and the lancet cap 106′ and the lancet body 104′ are integrally connected together by a weakened part 108′. As shown in FIGS. 23 and 26, the protective cover 102′ is provided to enclose a part of the lancet body 104′. Such lancet assembly 100′ is loaded into the injector 200′, and then the lancet cap 106′ is removed. By the removal of the lancet cap, the tip of the pricking needle 105′ is exposed, and thereby the lancet can serve to prick.

The injector 200′ shown in FIG. 24 can be used in combination with the lancet assembly 100′ to launch the lancet body with the tip of the pricking needle 105′ exposed. The injector 200′ ccmprises a plunger 204′ that is capable of engaging with a rear end portion of the lancet body to launch the lancet body in the pricking direction (see FIG. 27). As shown in FIG. 27, the lancet assembly 100′ is loaded into the injector 200′ by inserting the lancet assembly 100′ into the injector 200′ through a front end opening 214′ of the injector 200′. As shown in FIG. 28, when the lancet assembly is inserted to some degree, a rear portion 116′ of the lancet assembly 100′ is held by tips 264′ and 266′ of the plunger 204′. Subsequently, when the insertion of the lancet assembly is continued, the plunger 204′ is thrust backward so that the launching energy is stored. That is, the retraction of the plunger 204′ can compress a spring (not shown) provided in the plunger 204′. This means that, when the compression is released, the plunger instantly moves forward to launch the lancet. FIG. 29 shows the injector 200′ in the state where the plunger has retracted, and the launching energy is stored therein.

After the loading of the lancet assembly 100′ into the injector 200′ is completed, the lancet cap 106′ is removed to expose the tip of the pricking needle 105′. The removal of the lancet cap 106′ will be described as follows:

As shown in FIGS. 25 and 26, the lancet body 104′ and the lancet cap 106′ are integrally connected together by the weakened part 108′ disposed between the lancet body and the lancet cap. The weakened part 108′ is broken by rotating the lancet body 104′ and the lancet cap 106′ around the pricking needle in the reverse direction to each other (see FIG. 29 showing an embodiment of rotating the lancet cap in the direction “G”), whereby the removal of the lancet cap 106′ can be performed.

When the pricking operation is carried out, the front end opening 214′ of the injector 200′ is applied to a predetermined region to be pricked (for example, a finger tip). Subsequently, the press part 542′ of a trigger component 514′ is pushed. See FIG. 30. The pushing of the press part 542′ results in an instantaneous expansion of the compressed spring, and thereby forcing the plunger 204′ to move forwardly to prick the predetermined region with the pricking needle.

With respect to the pricking device as described above, there is a concern that the pricking needle which has been once used is re-used. That is, if the backward movement of the plunger 204′ after the pricking operation is made possible by any means, “ready state for pricking” as shown in FIG. 30 can be obtained again, which leads to the possibility that the used lancet is again used for another pricking.

The pricking device as described above makes use of a plate spring action of the trigger component 514′ to cease the engagement of the trigger component with the lancet body. It is thus necessary to surely push the press part 542′ in order to ensure the launching of the pricking needle. In this regard, there is a concern that the plate spring of the trigger component 514′ does not sufficiently function depending on which region of the press part 542′ is pressed.

The present invention has been devised in view of the above-mentioned circumstances. That is, an object of the present invention is to provide a pricking device which can prevent the re-use of the lancet which has been once used. Another object of the present invention is to provide a pricking device with an improved launching operability.

### Means for Solving the Problems

In order to achieve the above objects, the present invention provides a lancet pricking device comprising a lancet equipped with a pricking needle, and a trigger for launching the pricking needle;

wherein the trigger comprises a first trigger part and a second trigger part disposed such as to cover the first trigger part;

the lancet is in engagement with the first trigger part at a point in time before the pricking needle is launched; and

when the second trigger part is pushed for the launching of the pricking needle, the first trigger part is displaced by the second trigger part, and thereby ceasing the engagement of the lancet with the first trigger part to launch the pricking needle.

The present invention is characterized at least in that the lancet pricking device has a trigger mechanism composed of substantially two elements of the first trigger part and the second trigger part wherein the first trigger part is capable of being displaced, and the second trigger part is capable of causing the displacement of the first trigger part. In this trigger mechanism, the pushing of the second trigger part toward the inside of the device causes the first trigger part to be displaced, and thereby ceasing the engagement between the first trigger part and the lancet body.

As used in the present description and claims, the term “displaced”/“displacement” substantially means a change in position, arrangement, shape or the like of at least a part of the intended part due to an external force. For example, the term “displaced”/“displacement” can include the change in form or configuration such as movement, inclining, warping and the like.

In one preferred embodiment, a front end and a rear end of the first trigger part move in the reverse direction to each other during the displacement of the first trigger part. That is, one end of the first trigger part and the other end thereof move in the reverse direction to each other. For example, when the front end of the first trigger part is pressed inwardly due to contact of the first trigger part with the second trigger part, the front end of the first trigger part moves inwardly whereas the rear end of the first trigger part moves outwardly.

In one preferred embodiment, the lancet pricking device further comprises a “lancet case for housing therein the lancet”. In this embodiment, both of the first trigger part and the second trigger part are integrally formed with the lancet case.

It is preferred that each of the first trigger part and the second trigger part has an elongated shape. The elongated first and second trigger parts are arranged to be superimposed over each other. In this case, it is also preferred that the first trigger part has a pivot point at an inner side (inner surface) thereof, such that both of a front end and a tear end of the first trigger part serve as free ends. While on the other hand, it is preferred that the second trigger part has its rear end serving as a fixed end and its front end serving as a free end.

In one preferred embodiment, the first trigger part has, at its rear end, an engagement part “a” for securing the trigger, whereas the second trigger part has, at its inner side (inner surface), an engaged part “a” for securing the trigger. Upon the displacement of the first trigger part, the engagement part “a” of the first trigger part engages with the engaged part “a” of the second trigger part, and thereafter the first trigger part cannot be any longer returned to its original state of the pre-displacement thereof. Alternatively or additionally, the lancet pricking device may further comprises a trigger support connected to the first trigger part, in which case the first trigger part has, at its front end, an engagement part “b” for securing the trigger, and the trigger support comprises an engaged part “b” for securing the trigger. Upon the displacement of the first trigger part, the engagement part “b” of the first trigger part engages with the engaged part “b” of the trigger support, and thereafter the first trigger part cannot be any longer returned to its original state of the pre-displacement thereof. As a result, lancet pricking device of the present invention, after being used, cannot be any longer returned to the ready state for pricking because the first trigger part cannot be any longer returned to the initial state (i.e., the pre-displacement state of the first trigger part).

The trigger support, which is connected to the first trigger part, may be formed integrally with the lancet case. That is, the first trigger part may be integrally formed with the lancet case via the trigger support.

In one preferred embodiment, a lancet cap for covering the pricking needle is provided with a protrusion. While the pricking needle is covered with the lancet cap, the protrusion of the lancet cap is positioned directly under the second trigger part such as to be able to abut against the second trigger part, and thereby the displacement of the first trigger part is prevented even if the second trigger part is pressed from the outside. More preferably, the protrusion of the lancet cap is provided directly under the second trigger part such that the protrusion is positioned between the trigger support and the front end of the second trigger part. In such embodiment, after the removal of the lancet cap from the pricking needle, the second trigger part can be pushed inwardly by pressing it from the outside, and thereby causing the displacement of the first trigger part.

The present invention provides the lancet pricking device which is suitably equipped with the above trigger mechanism. Such lancet pricking device of the present invention is characterized in that it comprises a lancet, a launching spring and a lancet case that houses the lancet and the spring, wherein

the lancet comprises a lancet body, a lancet cap and a pricking needle in which the pricking needle is situated in both of the lancet body and the lancet cap;

a trigger integrally provided with the lancet case comprises a first trigger part and a second trigger part positioned such as to cover the first trigger part;

wherein the spring is attached to the lancet body, and the lancet body is in engagement with the first trigger part such that the spring is kept compressed before the pricking needle is launched; and

wherein, after the lancet cap is removed, the second trigger part can be pushed inwardly by pressing it from the outside to displace the first trigger part, and thereby ceasing the engagement of the lancet body with the first trigger part. Preferably, the lancet pricking device further comprises an exterior component located outside of the lancet case, namely, the device further comprises a component that encloses at least a part of the lancet case.

In such lancet pricking device, the locking of the lancet body is released by the trigger mechanism comprised of the first trigger part and the second trigger part, and thereby the “lancet body with the pricking needle exposed” is caused to be launched in the direction of pricking due to the expansion of the compressed launching spring. It is preferred that the lancet body is provided with an elastic portion. When the lancet body is launched in the direction of pricking, the elastic portion slides on the lancet case and/or an inner wall surface of the exterior component with the movement of the lancet body.

Furthermore, the present invention also provides a lancet pricking device with a desired attachment portion for a launching spring. Such lancet pricking device of the present invention is characterized in that the launching spring is attached to a spring-attachment portion provided at a rear-sided inner wall of the lancet case, and the spring-attachment portion has a bent form of an elongated part (particularly, a “generally tabular part having entirely a rectangular shape) with a free end and a fixed end. For example, the spring-attachment portion has a form of hook (e.g., fishing needle form or U-like form) composed of an elongated part with a free end and a fixed end. The spring-attachment portion can facilitate the assembly of the lancet pricking device. In particular, the spring-attachment portion can contribute to an easy attachment of the launching spring through an opening of the lancet case.

### Effect of the Invention

In accordance with the lancet pricking device of the present invention, once the pricking needle is launched, the first trigger part cannot be any longer returned to its original state of the pre-displacement. This makes it impossible it to engage the lancet with the first trigger part. Therefore, the device cannot restore its original state after the launching of the pricking needle. That is, the lancet pricking device of the present invention has such a structure that the pricking needle which has been already used for the pricking cannot be used again. Thus, the pricking device of the present invention does not have the risk of re-using the pricking needle, and is desirable from a hygiene and safety standpoint. The user has no choice but to use the lancet pricking device of the present invention only as a “non-reusable/disposable type device”, and thereby the hygiene for the subject of the blood sampling can be secured inevitably.

In the lancet pricking device of the present invention, when the second trigger part arranged to cover the first trigger part is pressed, the displacement of the first trigger part is effectively caused by a pressing action of the second trigger part. In accordance of the invention, any pressing region or point of such second trigger part can cause the displacement of the first trigger part. Thus, the launching of the pricking needle can be performed regardless of which region or point of the second trigger part is pressed, which is desirable from the viewpoint of the operability in use. That is, the present invention can provide the user-friendly pricking device, ensuring and facilitating the launching operation of the pricking needle. The lancet pricking device of the present invention has such a small size to enable a holding by the fingers, and thus actually has an improved operability.

Furthermore, the lancet pricking device of the present invention is substantially composed of three parts, i.e., “lancet”, “launching spring” and “lancet case”, or four parts, i.e., “lancet”, “launching spring”, “lancet case” and “exterior component”. Thus, the structure of the device is very simple and has the small size as a whole. In particular, the lancet pricking device of the present invention has such a relatively simple structure that the “lancet” and the “launching spring” are housed in the lancet case, and also the size of the lancet case, which size determines the entire size of the device, substantially corresponds to the small size of the lancet. As a result, the lancet pricking device of the present invention is not only easy to manufacture, but also excellent in a transport efficiency and a storage space thereof. Moreover, in a case where the device comprises the spring (launching spring)-attachment portion which has a bent form of an elongated part with a free end and a fixed end, the present invention makes it possible to achieve a simplified assembly of the lancet pricking device. This means that the lancet pricking device of the present invention is excellent also from the viewpoint of an assembly efficiency and a manufacturing efficiency thereof.

### BRIEF DESCRIPTION OF THE DRAWINGS

FIG. 1 is a schematic view of a lancet pricking device according to the present invention.

FIG. 2 is a schematic cross-sectional view showing “displacement” of a first trigger part.

FIG. 3 is a perspective view showing a trigger integrally formed with a lancet case.

FIG. 4 is a perspective view showing a trigger integrally formed with the lancet case (with a lancet housed therein).

FIG. 5 is a schematic cross-sectional view showing structural features of a first trigger part and a second trigger part.

FIG. 6 is a schematic cross-sectional view showing variations of trigger mechanism with time in a pushing operation.

FIG. 7 is a perspective view and a cross-sectional view showing an embodiment wherein a lancet cap has a protrusion.

FIG. 8 is a perspective view showing a lancet pricking device at a point in time when a pricking needle is covered with a lancet cap.

FIG. 9 is a perspective view showing a lancet pricking device at a point in time after a lancet cap is removed from a pricking needle.

FIG. 10 is a view of an appearance of a lancet pricking device according to the present invention.

FIG. 11 is an appearance view and an exploded perspective view of a lancet pricking device according to the present invention.

FIGS. 12(a) and 12(b) are a perspective view and a cross-sectional view showing an internal structure of a lancet pricking device according to the present invention.

FIGS. 13(a) to 13(c) are a perspective view and a side view of a lancet pricking device according to the present invention.

FIGS. 14(a) to 14(c) are a perspective view, a side view and a cross-sectional view of the lancet pricking device according to the present invention.

FIG. 15 is a perspective view of a launching spring used in the lancet pricking device.

FIG. 16 is a perspective view of an exterior component that can be used in the lancet pricking device.

FIGS. 17(a) to 17(c) are a perspective view and a cross-sectional view showing an internal structure of a lancet pricking device according to the present invention.

FIG. 18 schematically shows embodiments of a lancet pricking device at the point in time after the launching, while schematically showing “a decrease in inner diameter of a lancet case at its tapered portion”.

FIGS. 19(a) to 19(d) are cross-sectional views for mainly explaining a pricking depth adjusting mechanism at the time of pricking.

FIGS. 20(a) to 20(e) are perspective views schematically showing embodiments of a lancet pricking device when being used.

FIGS. 21(a) to 21(c) are schematic views showing variations of a spring-attachment part with time when assembling a lancet pricking device.

FIG. 22 is perspective views schematically showing a modified example of an appearance of a lancet pricking device according to the present invention.

FIG. 23 is a perspective view showing an appearance of a lancet assembly.

FIG. 24 is a perspective view showing an appearance of an injector.

FIG. 25 is a perspective view showing an appearance of a lancet.

FIG. 26 is a perspective view showing a lancet of FIG. 25, cut away in half so as to make it easy to understand the inside of the lancet.

FIG. 27 is a perspective view showing the state before a lancet assembly is loaded into an injector.

FIG. 28 is a perspective view showing the state in which a lancet is held by the tip of a plunger upon loading a lancet assembly.

FIG. 29 is a perspective view showing the state of completion of loading a lancet assembly wherein a plunger cannot be retracted any longer.

FIG. 30 is a perspective view sowing the state in which a lancet cap has been removed and thus a lancet is ready for pricking.

### MODES FOR CARRYING OUT THE INVENTION

A lancet pricking device according to the present invention will be described in detail with reference to the accompanying drawings.

The term “direction” as used throughout the claims and description is defined as follows: The direction in which the pricking needle is launched for pricking is regarded as a “forward” direction. The reverse direction thereto is regarded as a “backward” direction. The “forward direction” substantially means the direction in which the pricking needle moves toward the region to be pricked in the body of the subject of blood sampling, and corresponds to a “pricking direction”. The direction toward the inside or center of the lancet pricking device (especially, lancet case) is defined as an “inward direction” or “inside”, whereas the reverse direction thereto, from the inside or center of the lancet pricking device (especially, lancet case) outward, is defined as an “outward direction” or “outside”. Such terms “forward”, “backward”, “inside” and “outside” are illustrated in the drawings.

Trigger Mechanism of Lancet Pricking Device>

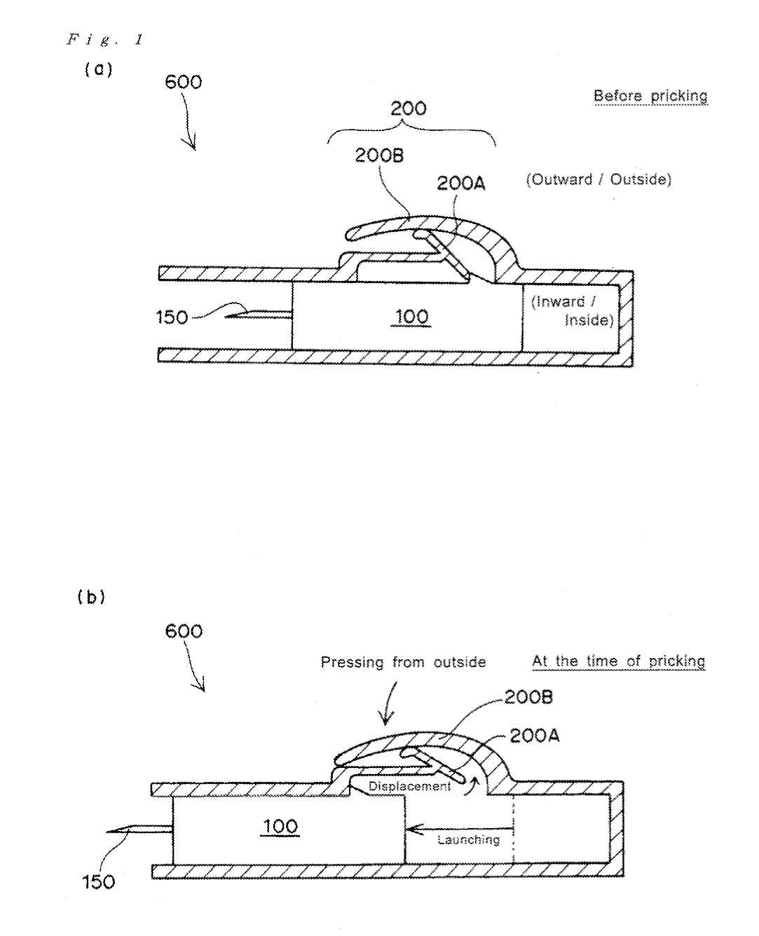

FIG. 1 shows a lancet pricking device 600 according to the present invention. As shown in FIG. 1, the lancet pricking device 600 comprises a lancet 100 equipped with a pricking needle 150, and a trigger 200 for launching the pricking needle.

The trigger 200 is substantially composed of two elements, i.e., a first trigger part 200A and a second trigger part 200B. The second trigger part 200B is located outside of the first trigger part, whereas the first trigger part 200A is located inside of the second trigger part. As shown in FIG. 1, the second trigger part 200B positioned outside is disposed such that it covers the first trigger part 200A positioned inside. That is, the second trigger part 200B is positioned outside the first trigger part 200A in such an arrangement that the first trigger part 200A and the second trigger part 200B are superimposed over each other.

It is preferred as shown in FIG. 1 that the first trigger part 200A and the second trigger part 200B are respectively in an elongated form. In order to cover the first trigger part 200A with the second trigger part 200B, the entire size of the second trigger part 200B is preferably larger than that of the first trigger part 200A. In this regard, the second trigger part 200B may have such a shape that its inner face defines a space for housing the first trigger part therein. For example, the second trigger part 200B may have a curved shape as a whole.

FIG. 1(a) shows the lancet pricking device at the point in time before a pricking needle is launched. As shown in FIG. 1(a), the lancet 100 is in engagement with the first trigger part 200A in the lancet pricking device 600 at the point in time before the launching. Specifically, the lancet 100 is in engagement with the first trigger part 200A so that the lancet 100 has a launching energy stored therein. For example, the lancet 100 is locked by the first trigger part 200A in such a way that the spring force or the like is applied to the lancet.

In order to launch the pricking needle, the second trigger part 200B is pushed from the outside. This causes the first trigger part 200A to be displaced, and thereby ceasing the engagement between the first trigger part 200A and the lancet 100 (see FIG. 1(b)). Such ceasing of the engagement can release the stored energy, and thereby the lancet 100 equipped with the pricking needle is launched in the direction of pricking.

Even by pressing random regions or points of the second trigger part 200B covering the whole of the first trigger part 200A, such trigger mechanism can cause the displacement of the first trigger part. Thus, the launching of the pricking needle can be surely performed regardless of which region or point of the second trigger part is pushed.

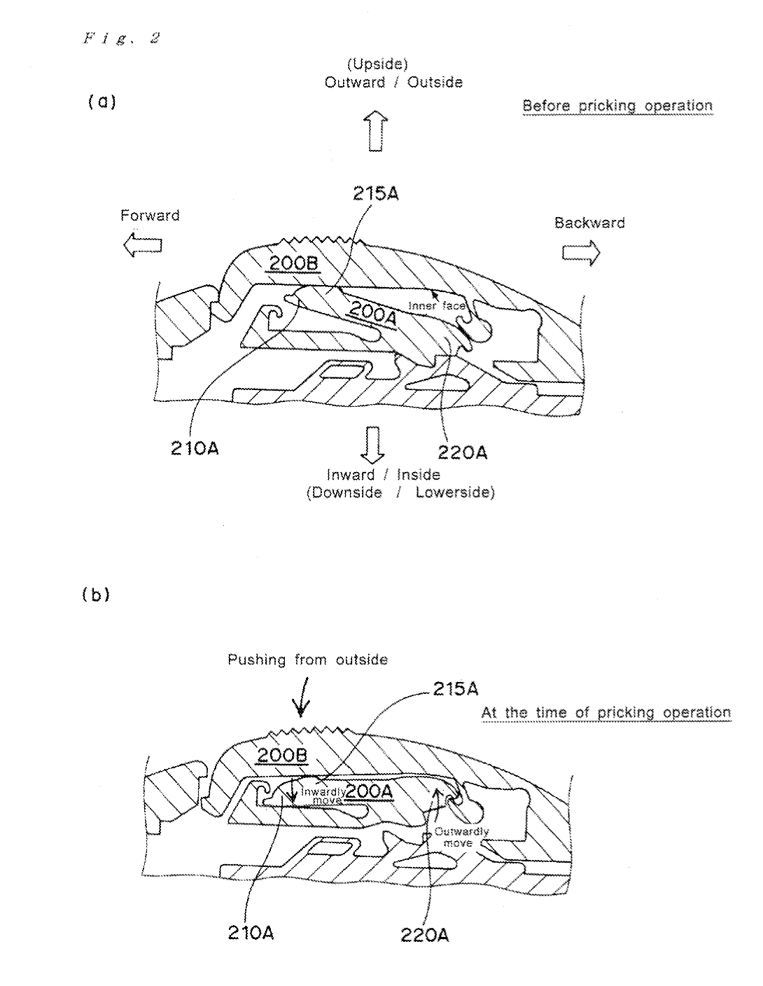

As for the displacement of the first trigger part, it is preferred that a front end 210A and a rear end 220A of the first trigger part 200A is caused to move in the reverse direction to each other, as shown in FIGS. 2(a) and 2(b). For example, it is preferred that the front end 210A of the first trigger part 200A moves inwardly, whereas the rear end 220A of the first trigger part 200A moves outwardly (see FIG. 2(b)). In other words, the first trigger part 200A preferably moves like a so-called “seesaw” such as to serve some point of the body of the first trigger part 200A as a pivot point. As shown in FIG. 2(a), the inner face of the second trigger part 200B is in contact with or in the vicinity of the front-side 215A of the first trigger part 200A. This facilitates the inward movement of the front end 210A of the first trigger part 200A and the outward movement of the rear end 220A thereof.

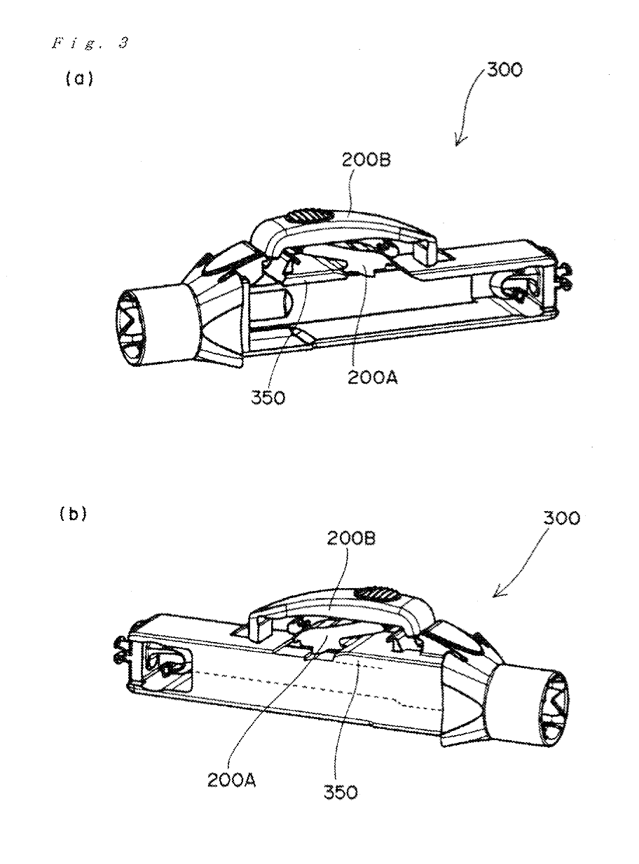

The lancet pricking device of the present invention preferably comprises a lancet case for housing the lancet therein. For example, the lancet pricking device comprises the lancet case 300 as shown in FIGS. 3 and 4. As can be seen from FIGS. 3 and 4, both of the first trigger part 200A and the second trigger part 200B are preferably integrally formed with the lancet case (especially, a main body of the case). In a case where the lancet case is made of resin, all of the first trigger part 200A, the second trigger part 200B and the lancet case 300 are preferably integrally formed with each other. It is preferred that a trigger support 350 is provided (note that the “trigger support” will be described in detail later). In this case, all of the first trigger part 200A, the second trigger part 200B, the trigger support 350 and the lancet case 300 are preferably integrally formed with each other.

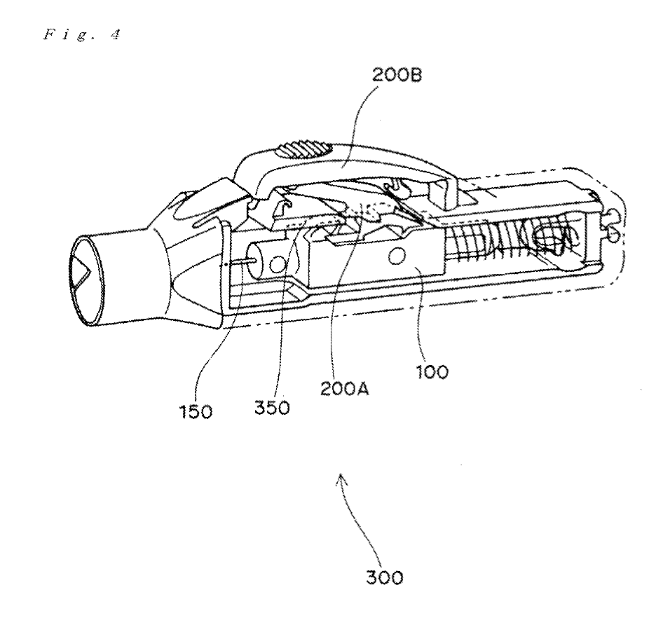

As can be seen from FIG. 5, it is preferred that the first trigger part 200A has, at its inner face, a pivot point 250A, and the front end 21′0A and the rear end 220A of the first trigger part 200A serve as free ends. While on the other hand, it is preferred that the rear end 220B of the second trigger part 200B serves as a fixed end, and the front end 210B of the second trigger part 200B serves as a free end. In this case, the displacement of the first trigger part 200A is effectively facilitated when the second trigger part 200B is pushed. Specifically, it is effectively facilitated that the front end 210A of the first trigger part 200A moves inwardly, whereas the rear end 220A of the trigger 200A moves outwardly. The inward moving distance of the front end 210A may be different from the outward moving distance of the rear end 220A, which may depend on the position of the pivot point 250A of the first trigger part 200A, for instance. As shown in FIG. 5, the pivot point 250A is provided at the rear side of the first trigger part 200A, and thereby the “inward moving distance of the front end 210A” can be larger than the “outward moving distance of the rear end 220A”.

The lancet pricking device of the present invention preferably has a function of securing the trigger by engagement. Specifically, the pricking device preferably has such a securing function that it can maintain the displaced state of the first trigger part (particularly, keep the displaced state of the first trigger part through the engagement) after the displacement of the first trigger part upon the pricking operation.

For example, as shown in FIG. 6(a), the first trigger part 200A has, at its rear end 220A, an engagement part “a” (225A) for securing the trigger, whereas the second trigger part 200B has, at its inner face 201B, an engaged part “a” (240B) for securing the trigger. In this case, after the displacement of the first trigger part 200A is caused, the engagement part “a” (225A) of the first trigger part 200A and the engaged part “a” (240B) of the second trigger part 200B engage with each other. Specifically, when the second trigger part 200B is pushed inwardly, the front end 210A of the first trigger part 200A moves inwardly, whereas the rear end 220A of the first trigger part 200A moves outwardly (see. FIG. 6(b)). As a result, the engagement part “a” (225A) of the first trigger part 200A engages with the engaged part “a” (240B) of the second trigger part 200B. This engagement serves to fix the displaced first trigger part 200A in place (see FIG. 6(c)), whereby the first trigger part 200A cannot be any longer returned to its original state which corresponds to the pre-displacement state of the trigger part 200A. In order to effectively facilitate the engagement of the first and second trigger parts, the engagement part “a” (225A) of the first trigger part 200A and the engaged part “a” (240B) of the second trigger part 200B preferably have complementary shapes to each other (as shown in FIG. 6, the engagement part “a” (225A) and the engaged part “a” (240B) may respectively have a hook shape, for instance).

In a case where the lancet pricking device has a “trigger support 350” connected to the first trigger part 200A as shown in the FIG. 6(a), the first trigger part 200A may have, at its front end 210A, an engagement part “b” (255A) for securing the trigger, whereas the trigger support 350 may have an engaged part “b” (355) for securing the trigger. In this case, after the displacement of the first trigger part 200A is caused, the engagement part “b” (255A) of the first trigger part 200A and the engaged part “b” (355) of the trigger support 350 engage with each other. Specifically, when the second trigger part 200B is pushed inwardly, the front end 210A of the first trigger part 200A moves inwardly (see FIG. 6(b)), and thereby the engagement part “b” (255A) of the first trigger part 200A engages with the engaged part “b” (355) of the trigger support 350. The engagement servers to fix the displaced first trigger part 200A in place (see FIG. 6(c)), whereby the first trigger part 200A cannot be any longer returned to its original state which corresponds to the pre-displacement state of the trigger part 200A. In order to effectively facilitate the engagement of the first trigger part and the support, it is preferred that the engaged part “b” (355) has a hook shape, whereas the engagement part “b” (255A) has a protruding shape at the tip of the first trigger part 200A (see FIG. 6(a)). As shown in FIGS. 3 and 6, the trigger support 350 is connected to the first trigger part 200A, and serves to support or fix the first trigger part 200A with respect to the lancet case 300. In particular, the connecting part or joint part between the first trigger part 200A and the trigger support 350 corresponds to the above “pivot point 250A”. Such trigger support 350 may have any suitable forms as long as it supports or fixes the first trigger part 200A and also can provide the “pivot point 250A”.

In the lancet pricking device having a function of securing the trigger by engagement, the displaced first trigger part, after the launching of the pricking needle, cannot be returned to its original state of the pre-displacement. This means that the lancet cannot any longer engage with the first trigger part after the launching. That is, the present invention makes it impossible to return the used pricking device to the state before the pricking. This eliminates the risk of re-use of the used pricking needle, which is desirable in terms of hygiene and safety. In light of the device having such re-use preventing mechanism, the lancet pricking device of the present invention can also be referred to as “single-use device”.

As shown in an enlarged view of FIG. 6(b), when the displaced first trigger part 200A upon pushing the trigger finally comes into contact with the trigger support 350. More specifically, when the front end 210A of the first trigger part 200A moves inwardly, an inner side (lower face) 212A of the front end 210A abuts against the outer side (upper face) of the trigger support 350. This restricts the degree of pushing the trigger at the time of the pricking operation, and thus can achieve a desirable trigger pushing operation. In other words, due to the effect that the first trigger part 200A abuts against the trigger support 350 in the pushing operation of trigger, which can prevent the excessive displacement of the first trigger part, that is, the excessive pushing of the second trigger part.

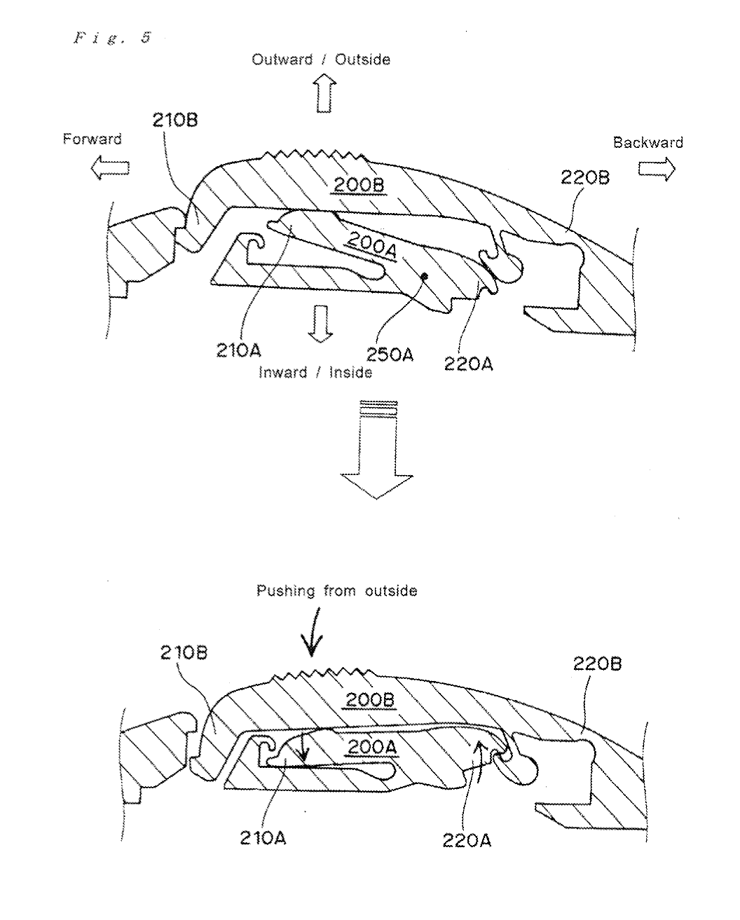

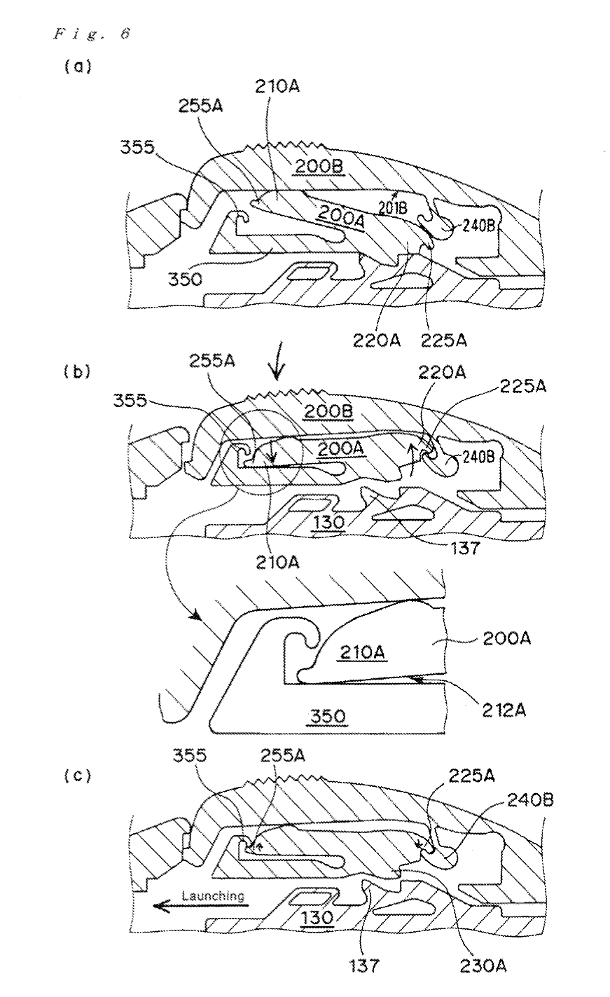

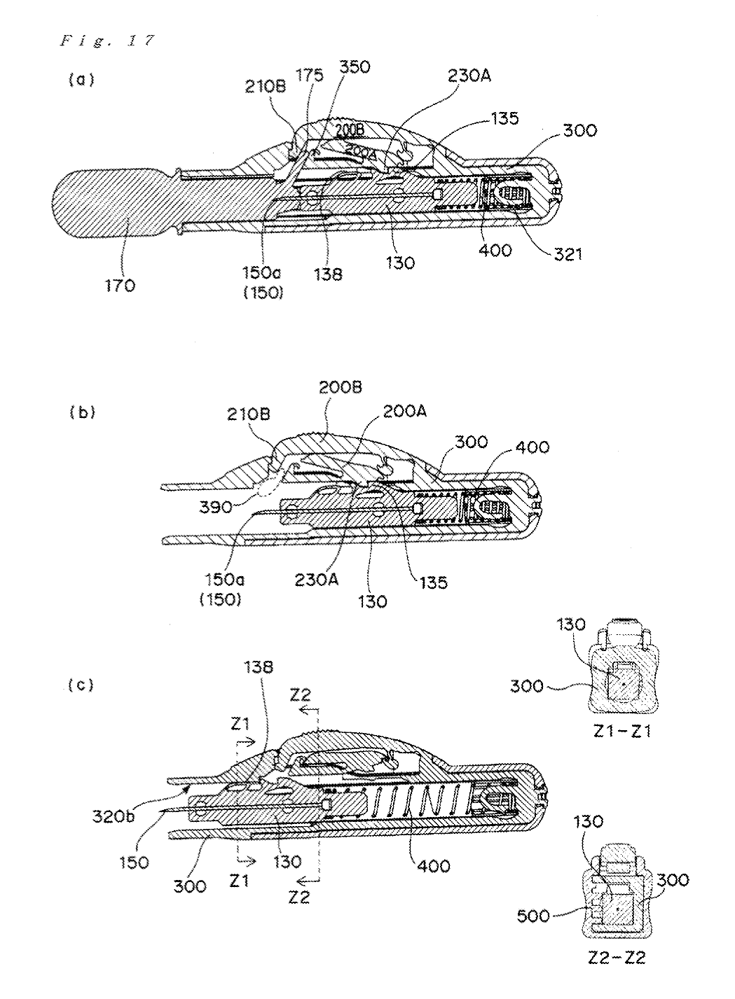

The lancet pricking device of the present invention preferably has a protrusion 175 disposed at a lancet cap 170 for covering the pricking needle 150 (particularly, a tip 150a of the pricking needle) as shown in FIGS. 7(a) to 7(c). In this case, as shown in FIG. 8, a protrusion 175 of the lancet cap 170 is preferably positioned directly under (directly inside) the second trigger part 200B such that the protrusion 175 can abut against the second trigger part 200B. For example, the uppermost surface of the protrusion is positioned under the inner surface of the second trigger part, preferably by 0.1 to 2 mm, more preferably, by 0.1 to 1 mm, and most preferably, by 0.1 to 0.5 mm. In particular, the protrusion 175 of the lancet cap 170 is preferably positioned between the front end 210B of the second trigger part 200B and the trigger support 350 (also see FIG. 17(a)). As seen from the embodiment shown in FIGS. 8 and 17(a), the protrusion 175 of the lancet cap 170 is sandwiched between the “front end 210B of the second trigger part 200B” and the “trigger support 350” so as to be brought into contact with them. This prevents the protrusion 175 from being substantially deformed or displaced even if the external force from the outside is applied thereto. That is, even when the second trigger part is pushed, followed by the abutting of the second trigger part onto the protrusion 175, the protrusion is not substantially deformed or displaced, and thereby the second trigger part 200B is effectively prevented from being further pushed. This results in substantially no displacement of the first trigger part 200A. In other words, when the protrusion 175 of the lancet cap 170 is located between the front end 210B of the second trigger part 200B and the trigger support 350, this causes no ceasing of the engagement between the first trigger part 200A and the lancet 100. On the other hand, as shown in FIGS. 9 and 17(b), when the lancet cap 170 is removed, the protrusion 175, which is provided in the cap, is also removed. This means that the protrusion 175 serving as “movement inhibition component” for the second trigger part 200B is eliminated. The removal of the protrusion 175 generates a space (particularly see “space” indicated by reference numeral 390 in FIG. 17(b)) under the second trigger part 200B (in particular, under a free end 210B of the front side end thereof). Because of such space, the second trigger part 200B (especially, the front end 210B thereof) can be pushed into the device to cause the substantial displacement of the first trigger part 200A. That is, the removal of the lancet cap 170 from the pricking needle 150 can contribute to a releasing of the locking between the first trigger part 200A and the lancet 100, so that the pricking needle can be launched.

### Embodiments of Lancet Pricking Device

Now, a lancet pricking device which is suitably equipped with the trigger mechanism according to the present invention will be described.

### (Basic Structure)

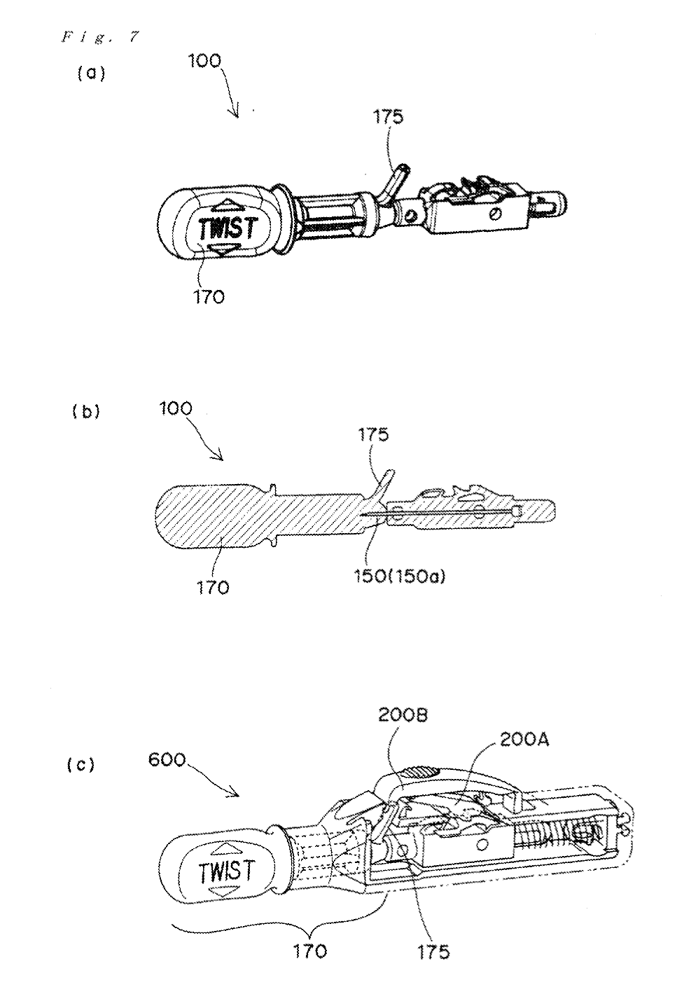

FIGS. 10 and 11 show a lancet pricking device 600 according to the present invention. FIG. 10 shows an external appearance of the lancet pricking device 600. FIG. 11 shows an exploded view and a development view of the lancet pricking device 600. As shown in FIG. 11, the lancet pricking device 600 according to the present invention is mainly composed of “lancet 100”, “launching spring 400”, the “lancet case 300 equipped with the trigger 200” and an “exterior component 500”.

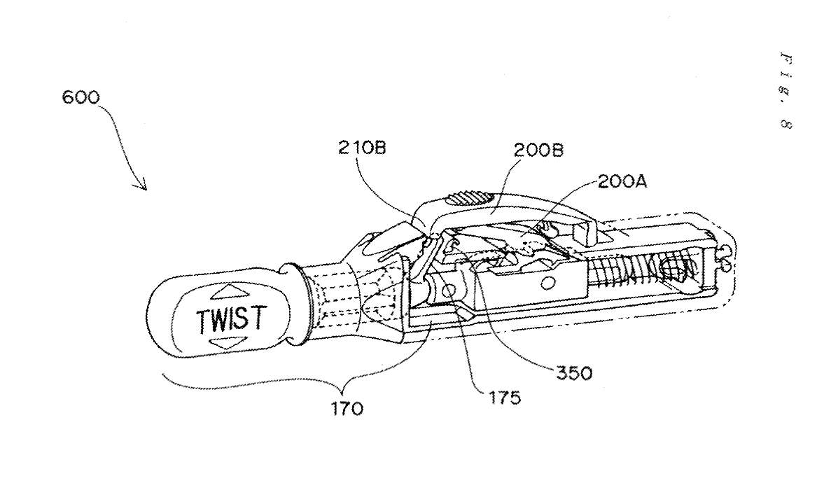

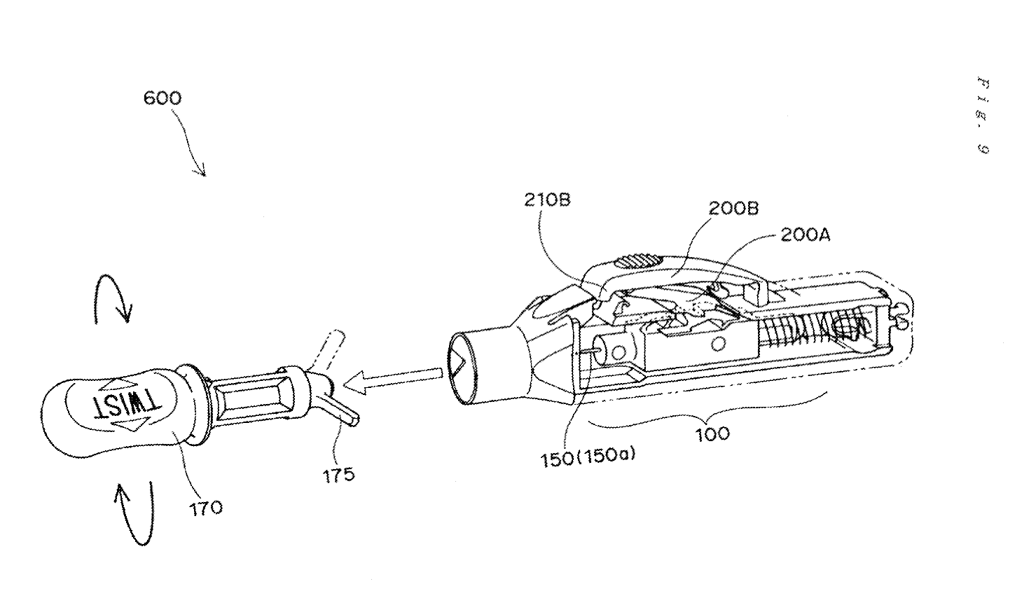

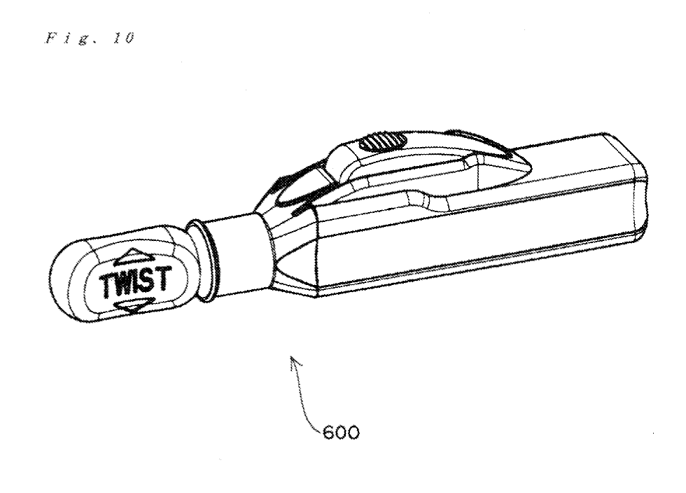

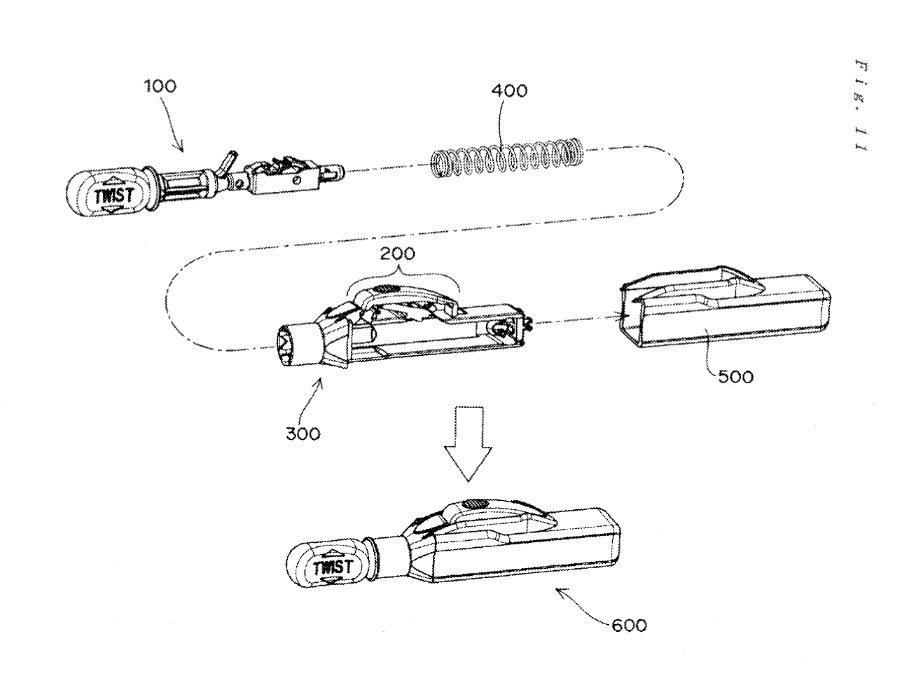

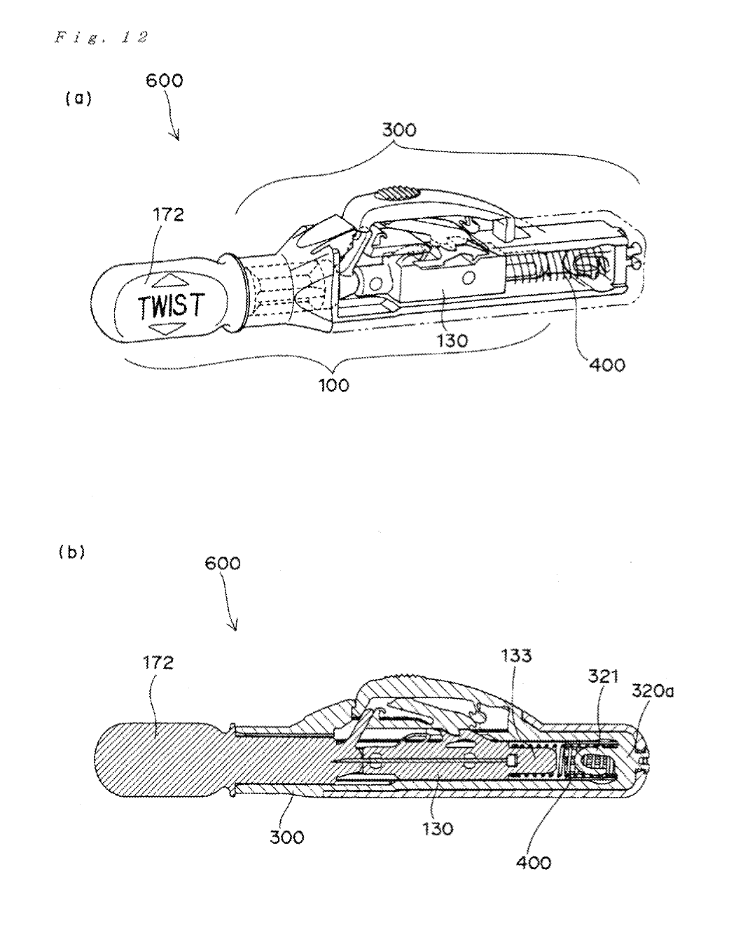

As shown in FIG. 12, the lancet pricking device 600 of the present invention has such a structure that the lancet 100 and the launching spring 400 are housed in the lancet case 300. Specifically, as shown in FIG. 12, the lancet 100 (except for a holding portion 172) and the launching spring 400 are housed in the lancet case 300 such that the spring 400 is held between the lancet 100 and the inner wall surface of the lancet case 300. More specifically, as shown in FIG. 12(b), one end of the spring 400 is attached to a rear end 133 of the lancet 100 in the lancet case 300, and the other end of the spring 400 is attached to a fitting portion 321 (i.e., “spring-attachment portion” to be described later) provided at a rear end-sided inner wall surface 320a of the lancet case 300. As can be seen from the comparison between FIG. 11 and FIG. 12, the launching spring 400 of the lancet case 300 is in a compressed state between the “lancet 100” and the “rear end-sided inner wall of the lancet case 300”. In other words, in the lancet pricking device 600 of the present invention, a lancet body 1301s locked in the lancet case 300 such that the launching spring 400 attached to the lancet body 130 is kept compressed.

In the following, components or parts regarding the lancet pricking device 600 will be described.

### (Lancet Case)

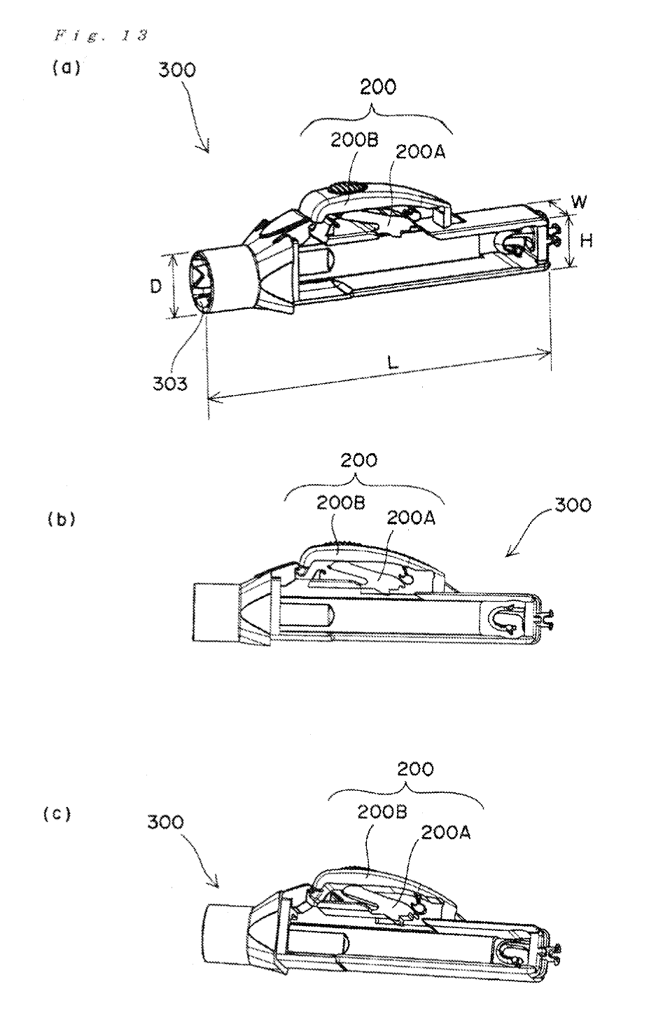

The lancet case 300 has, as a whole, a shape of square box or square tube as shown in FIG. 13, for example. The lancet case 300 has small dimension. For example, the dimensions (L, H, W, D) shown in FIG. 13(a) regarding the lancet case may be as follows: “L” is in the range of 35 to 50 mm (for example, about 37 mm), “H” is in the range of 4 to 10 mm (for example, about 5 mm), “W” is in the range of 4 to 10 mm (for example, about 5 mm) and “D” is in the range of 4 to 11 mm (for example, about 6 mm). However, the shape of the lancet case 300 is not limited to the square box or square tube, and may be a cylinder. The lancet case 300 may be formed of any kind of resin material which is used for lancets in general. As shown in FIG. 13, the lancet case 300 is provided with an opening end 303. The opening end 303 serves as not only a portion into which the lancet 100 and the launching spring 400 can be inserted and thus set upon assembly of the pricking device, but also a portion applied to the region (e.g., finger) to be pricked at the time of pricking operation.

The lancet case 300 is provided with the trigger 200 composed of the first trigger part 200A (inner side trigger part) and the second trigger part 2008 (outer side trigger part) (see FIG. 13). As described above, the trigger 200 is used for the pricking operation. When pressed from the outside, the trigger 200 functions to launch the pricking needle (more specifically, launch the lancet body equipped with the pricking needle). That is, when the second trigger part 200B is pressed from the outside so as to launch the pricking needle, the displacement of the first trigger part 200A is caused, and thereby ceasing the engagement between the first trigger part 200A and the lancet 100. The basic structure and function of the trigger itself have been already described above, and thus a repeated description will be omitted here.

### (Lancet)

The lancet 100 used in the lancet pricking device 600 is shown in the perspective view, the side view and the cross-sectional view in FIG. 14. Similarly to the lancet case 300, the lancet 100 is also small, and has such a size as to be housed in the lancet case 300. For example, the dimensions (L₁, L₂, H₁, H₂, W) shown in FIG. 14 regarding the lancet may be as follows: “L₁” is in the range of 40 to 55 mm (for example, about 43 nm), “L₂” is in the range of 16 to 21 mm (for example, about 18 mm), “H₁” is in the range of 2 to 10 mm (for example, about 3.5 mm), “H₂” is in the range of 2 to 12 mm (for example, 6.5 mm) and “W” is in the range of 2 to 12 mm (for example, about 3.5 mm). As shown in FIG. 14, the lancet 100 comprises the lancet body 130, the lancet cap 170 and the pricking needle 150 (specifically, see FIG. 14(c) as to the pricking needle 150). The pricking needle 150 is, for example, a metal needle. The pricking needle 150 is situated in both of the resin lancet body 130 and the resin lancet cap 170 wherein the tip 150a of the pricking needle 150 is covered with the lancet cap 170, as shown in the cross-sectional view of FIG. 14(c). It is preferred that the lancet body 130 and the lancet cap 170 are integrally connected to each other via only a small contact portion. The lancet 100 can be formed of resin (such as polyethylene (e.g., LDPE), polypropylene or the like) by inserting the pricking needle 150 into a die, in a so-called insert molding process. In this case, the contact portion can be formed upon carrying out the insert molding process. Accordingly, the contact portion can be formed of the same resin as that of the lancet cap 170 and the lancet body 130. The contact portion is required to be broken upon removing the lancet cap. Thus, the contact portion can be referred to as a “weakened portion” or “easily broken portion”. The contact portion may have a notch so that the contact portion can be easily broken. In some cases, the contact portion may be cut off in advance. No contact portion may also be provided in the lancet. As long as the lancet cap can be “twisted” to expose the tip 150a of the pricking needle 150 in the lancet body 130, the form of the contact portion is not limited to the specific one.

The lancet cap 170 has the protrusion 175 extending backwardly. The protrusion 175 preferably has a plate-like shape as shown in FIG. 14. When the lancet 100 with the protrusion is disposed in the lancet case 300 (that is, in the lancet pricking device at the point in time before the pricking operation), the protrusion 175 is positioned directly under the second trigger part 200B such as to be able to abut against the second trigger part 200B. It is particularly preferred that the protrusion 175 of the lancet cap 170 is positioned between the trigger support 350 and the front end 210B of the second trigger part 200B (see FIG. 8 and FIG. 17(a)).

The body of the pricing needle 150 is fixed in the lancet body 130. At the time of the pricking operation, the lancet body 130 is launched forward together with the pricking needle 150. As shown in FIG. 14(a), a spring-attachment portion 133 to which the launching spring is attached is provided at the rear end of the lancet body 130. The lancet body 130 is provided with a “locking portion 135 for securing the lancet to the inside of the lancet case” at its upper side. The lancet body 130 is also provided with “claw portion 137 for defining or adjusting the pricking depth upon the pricking”.

The lancet body 130 used in the present invention is preferably provided with an elastic portion 138. The elastic portion 138 can improve the straight shot of the pricking needle 150 upon the pricking (namely improve a straight pricking pathway). The elastic portion 138 can be deformed by applying the external force thereto. The elastic portion 138 may have any form as long as it is returned to its “original state” or “state similar to the original state” after removing the applied external force therefrom. For example, the elastic portion 138 has a cavity 138a therein as shown in FIGS. 14(a) to 14(c) (in particular, FIG. 14(b)). That is, the elastic portion may have a hollow structure. It is preferred that the elastic portion is made of resin. The resin elastic portion may be formed of any kind of resin material that is used for lancets in general. For instance, the elastic portion may be formed of resin material such as polyethylene and polypropylene. When the external force from the outside is applied, the elastic portion with the hollow structure can be compressed so as to reduce its size in the applied direction of the external force. When being released from the external force, the elastic portion can be returned to its original shape. The cavity 138a of the elastic portion with the hollow structure is not limited to the specific one shown in the figures. For example, the cavity 138a may have a slit-like shape elongated in the direction of pricking, or may have a flat shape (in other words, a rectangular cross-section shape) or the like.

### (Launching Spring)

The launching spring 400 is shown in the perspective view in FIG. 15. The launching spring 400 serves to shoot, fire or launch the needle as suggested by its name. In other words, the launching spring 400 is one that generates an impellent force for launching the pricking needle (i.e., “lancet body with the pricking needle exposed”) or a drive force for pricking. As described above, the compressed spring 400 is provided between the “rear end-sided inner wall surface 320a of the lancet case 300” and the “rear end attachment portion 133 of the lancet 100” (see FIG. 12(b)). The launching spring 400 is preferably made of metal. For instance, the spring 400 may be a metallic coil spring. The size of the launching spring 400 is not limited to a specific one as long as it can be housed in the lancet case. For example, the launching spring 400 may have a length (L₃, D), at the point in time when it is not compressed as shown in FIG. 15, of L₃=12 to 20 mm (for example, about 18 mm) and D=2 to 10 mm (for example, about 2 mm).

### (Exterior Component)

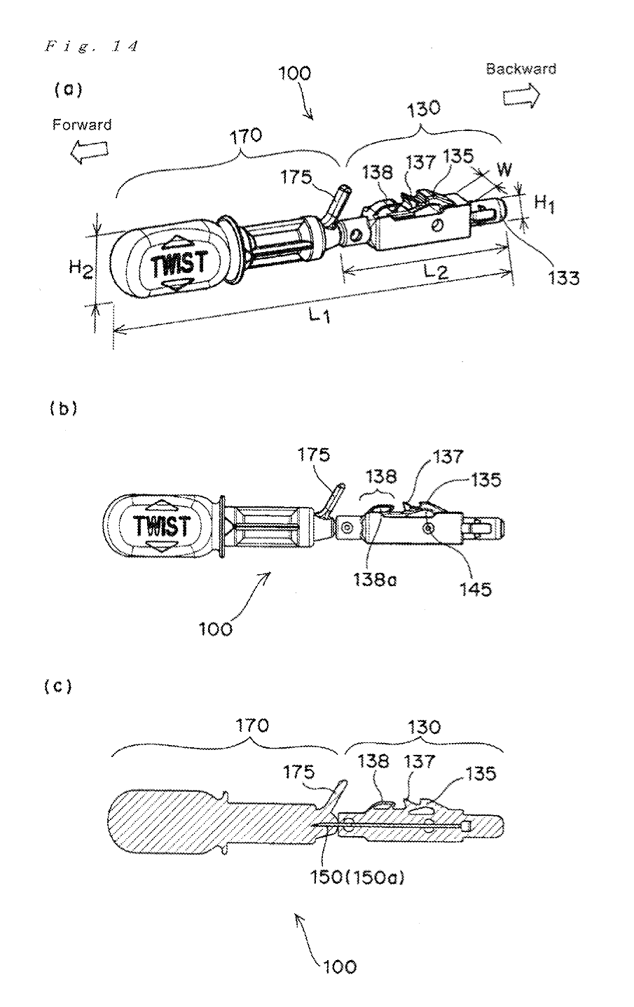

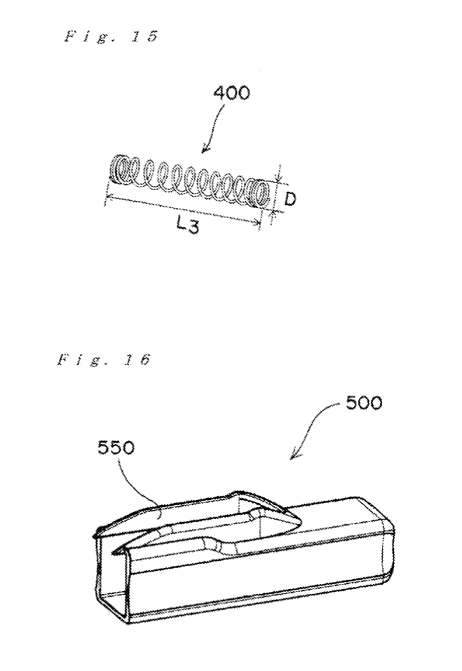

The exterior component 500 is shown in the perspective view in FIG. 16. The exterior component 500 is an outer wearable component, as suggested by its name. The component is not always necessary from the viewpoint of the present invention. However, the provision of the exterior component shown in FIG. 16 is preferable because the integrity of the entire device is provided, or because the lancet can be effectively held by the lancet case. As can be seen from the embodiment shown in FIG. 16, the exterior component 500 has the structure which at least partly encloses the lancet case 300. For example, the exterior component 500 may have a cutout 550 at its portion corresponding to the position of the trigger.

### <Entire Structure and Function of Lancet Pricking Device>

### (Assembly of Pricking Device and Locking of Lancet Body)

In the lancet pricking device 600 of the present invention, as shown in FIG. 17(a), the lancet body 130 is in engagement with the lancet case 300 such that the compressed state of the launching spring 400 is maintained. Specifically, the lancet body 130 is in engagement with the first trigger part 200A integrally formed with the lancet case 300. Such lancet pricking device 600 is assembled by inserting the lancet 100 and the launching spring 400 from the opening end 303 into the lancet case 300, followed by securing the lancet 100 to the lancet case with the launching spring 400 thrust therein (see FIG. 11). When the lancet is inserted into the lancet case 300, a warp of a hollow portion 145 of the lancet 100 (see FIG. 14(b)) is caused, and thereby the insertion of the lancet is facilitated.

In a case where the spring attachment portion 321 is comprised of a bent elongated part having a free end and a fixed end, the assembly of the lancet pricking device (in particular, attaching of the launching spring 400) is effectively facilitated. That is, as shown in FIGS. 17(a) and 21, when the spring attachment portion 321 is provided in the form of a hook (e.g., fishing-needle form or U-like form), the inward warp of the spring attachment portion 321 can be easily performed (see FIGS. 21(a) and 21(b)), so that the engagement of the launching spring coil with the attachment portion can be effectively facilitated. It is preferred that the spring attachment portion 321 has a protrusion 321a that protrudes outwardly. In particular, a first protrusion 321a₁ is preferably provided at the free end of the bent elongated part, whereas a second protrusion 321a₂ is preferably provided at the fixed end of the bent elongated part as shown in FIG. 21(a). In this case, once the spring 400 increases its coil diameter to ride over the protrusion 321a, then the spring 400 is returned to have its original coil diameter. As a result, a joint between the spring 400 and the spring attachment portion 321 cannot be easily released any longer (see FIGS. 21(a) to 21(c)).

As shown in FIG. 17(a), the locking (engagement) of the lancet body with the lancet case is performed by abutting an engaging portion 135 of the lancet body 130 against an engaged portion 230A of the first trigger part 200A. That is, the engaging portion 135 of the lancet body 130 abuts against the engaged portion 230A of the first trigger part 200A while the lancet 100 is thrust backwardly inside the lancet case 300 so as to compress the launching spring 400. In the lancet case 300, the forward force attributed to the compressed spring 400 acts on the lancet 100, and thereby the lancet 100 is forced to forwardly move. However, the forward movement of the lancet body 130 is locked by the engagement of its engaging portion 135 with the engaged portion 230A of the first trigger part 200A integrally formed with the lancet case 300. It should be noted that the lancet body 130 is locked or secured to the lancet case 300, whereas the lancet cap 170 is not locked or secured to the lancet case 300, and thereby the lancet cap 170 can be removed upon the pricking operation.

As shown in FIG. 17(a), while the tip 150a of the pricking needle is covered with the lancet cap 170, the protrusion 175 of the cap is positioned directly under the second trigger part 200B such as to be able to abut against the second trigger part 200B. More specifically, as shown in FIG. 17(a), at least a part of the protrusion 175 of the cap is positioned in a space formed between the “front end 210B of the second trigger part 200B” and the “trigger support 350”. As can be seen from the embodiments shown in FIG. 17, the protrusion 175 of the lancet cap 170 is sandwiched between the “front end 210B of the second trigger part 200B” and the “trigger support 350” so as to be brought into contact with the front end and the support. This results in substantially no deforming or displacement of the protrusion 175 in spite of the external force from the outside. Thus, even if the second trigger part 200B is pushed into the case, the protrusion 175 suitably serves as an inhibitor, and thereby the displacement of the first trigger part 200A is prevented. As a result, there is no ceasing between “engaging portion 135 of the lancet body 130” and the “engaged portion 230A of the first trigger part 200A”. In contrast, when the lancet cap 170 is removed as shown in FIG. 17(b), the protrusion 175 of the cap is also removed. This means that the inhibitor for the movement of the second trigger part 200B is eliminated. As shown in FIG. 17(b), there is generated a space 390 under the second trigger part 200B (especially, front free end 210B of the second trigger part). In this case, when the second trigger part 200B is pushed toward the inside of the lancet case, the displacement of the first trigger part 200A is caused. This makes it possible to cease the engagement between the “engaging portion 135 of the lancet body 130” and the “engaged portion 230A of the first trigger part 200A”. Such cease of the engagement causes the compressed spring 400 to expand, and thereby forcing the “lancet body with the pricking needle exposed” to be launched in the pricking direction.

The “lancet body with the pricking needle exposed” is launched such that the pricking needle moves substantially along the center axis of the lancet case. This means that “the lancet body with the pricking needle exposed”, after being launched, moves in the pricking direction along the inner wall of the lancet case. In other words, the lancet body moves forwardly in such a manner as to be guided by the inner wall surface of the lancet case. In this regard, as shown in FIG. 17(c), the lancet body 130 moving the in the pricking direction is surrounded by the inner wall surface(s) of lancet case 300 and/or the exterior component 500.

### (Improved Straight Pricking Pathway)

The “improved straight pricking pathway” of the lancet pricking device 600 will be described below. It is preferred in the lancet pricking device 600 of the present invention that the lancet body 130 is provided with the elastic portion 138 as described above (see, for example, FIGS. 17(a) to 17(c)). The provision of the elastic portion 138 can improve the straight pricking movement of the pricking needle 150 upon the pricking operation. The term “elastic portion” as used herein means that a member, part or portion capable of being deformed by applying the external force thereto, and also capable of being returned to its “original form” or “similar form to the original one” after removing the external force.

When the lancet body 130 equipped with the elastic portion 138 is launched to move in the pricking direction, the elastic portion 138 of the moving lancet body 130 slides on the inner wall surface 320b of the lancet case 300 (see FIG. 17(c)). That is, when the lancet body with the pricking needle moves in the pricking direction, the elastic portion provided in the lancet body also moves in the pricking direction in the same manner. During the movement of the lancet body, the elastic portion comes into contact with the inner wall surface of the lancet case. As shown in FIG. 18, the elastic portion 138 (its hollow portion or cavity 138a) is preferably deformed due to the contact of the elastic portion with the inner wall surface of the lancet case. In particular, the elastic portion is brought into contact with the inner wall surface of the lancet case so as to receive the external force due to the inner wall surface of the lancet case. Thus, the elastic portion can absorb the shock occurred in the launched pricking needle, so that the pricking needle moving in the pricking direction is stabilized. In other words, the moving elastic portion slides on the inner wall surface of the lancet case, and thus the stress generated in the pricking needle can be relieved by a cushion effect of the elastic portion, even though the pricking needle receives some transverse force. As a result, a linear pricking pathway of the needle can be ensured. The inner wall surface 320b of the lancet case 300 may be provided with a tapered portion (i.e., “sloped portion”). In this case, the elastic portion 138 can effectively receive the force when it slides on the tapered portion. That is, the moving elastic portion tends to be easily warped by the tapered portion, so that the shock of the lancet body can be effectively absorbed by the elastic portion upon the pricking operation, which can further stabilize the pricking pathway of the needle. In the case where the inner wall surface 320b of the lancet case 300 has the tapered portion, a decrease ratio “R” of inner diameter of the lancet case, which is indicated by the following formula, is preferably in the range of about 0.1 to 20%, more preferably in the range of about 0.1 to 15%, and most preferably in the range of about 0.1 to 10%.

R=(D1−D2)/D1×100

where R(%) is a decrease ratio of inner diameter of the lancet case, attributed to the tapered portion,

D1 (mm) is an inner diameter of the case, the diameter being positioned at the rear side with respect to the tapered portion (see FIG. 18), and

D2 (mm) is an inner diameter of the case, the diameter being narrowed by the tapered portion (see FIG. 18).

The phrase “improved straight pricking pathway” means that the pricking pathway of the needle becomes substantially constant even when being used by a different user. Accordingly there is provided such an advantageous effect that the fluctuations of the pricking pathway, attributed to the different users, can be effectively reduced.

### (Pricking Depth Adjustment Mechanism)

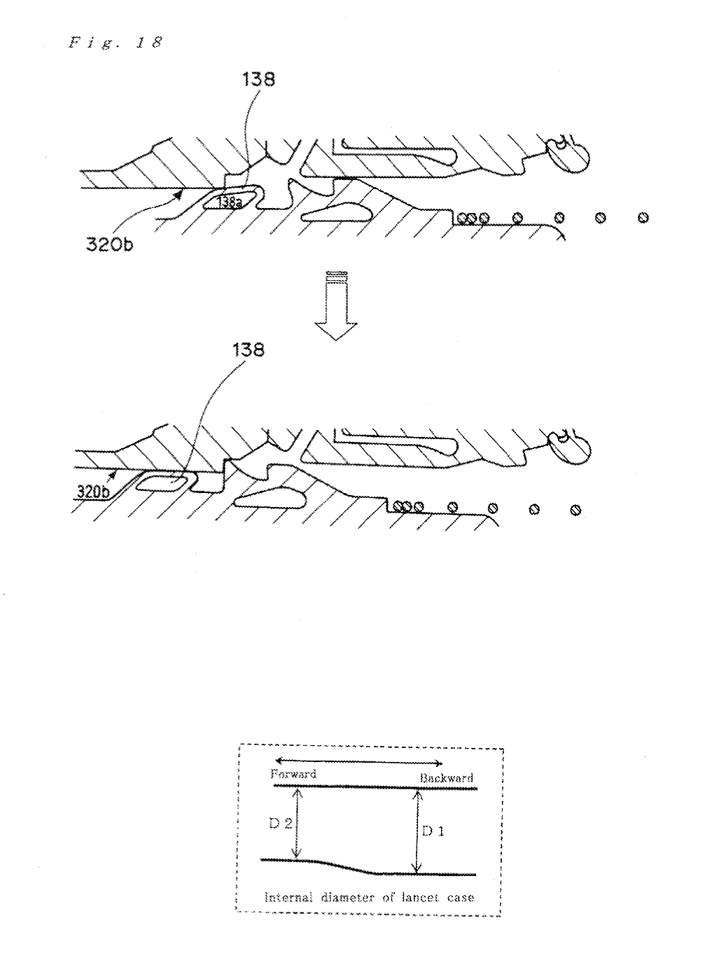

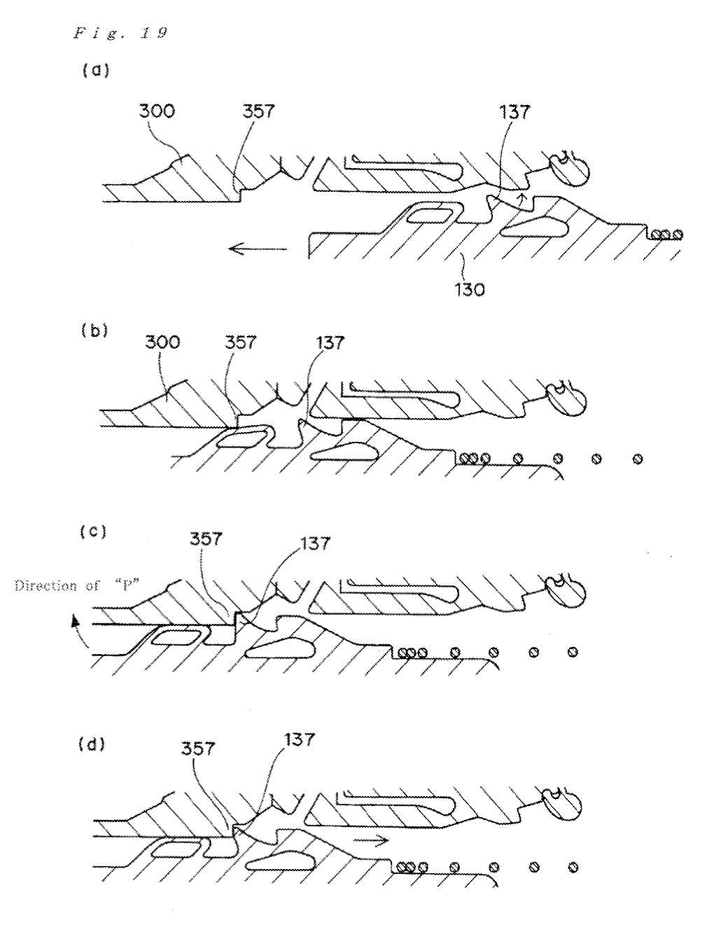

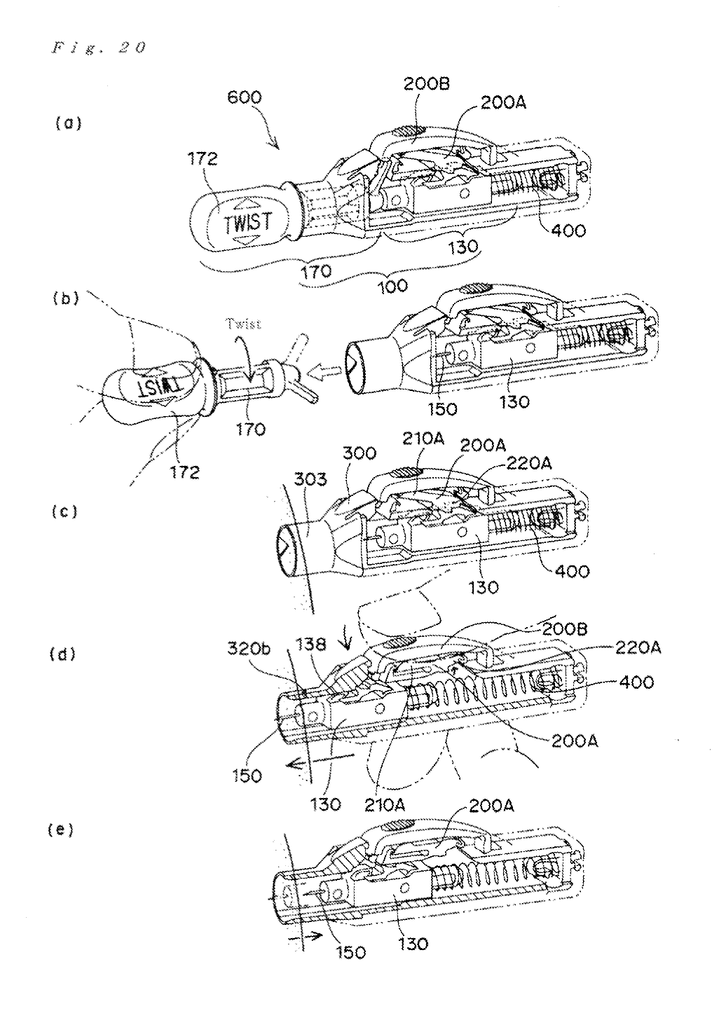

The lancet pricking device 600 of the present invention preferably has a pricking depth adjustment mechanism. Specifically, as shown in FIG. 19, a claw portion 137 for defining the pricking depth is provided in the lancet body 130, whereas a stepped portion 357 for defining the pricking depth is provided in the lancet case 300. As shown in FIGS. 19(a) to 19(d), the claw portion 137 of the lancet body makes contact with or hit the stepped part 357 provided at the inner wall of the lancet case 300 upon the pricking operation, whereby the length of the pricking needle protruding from the open end of the lancet case is defined or limited. That is, the forward movement of the pricking needle is restricted by the collision between the claw portion 137 and the stepped portion 357, making it possible to define the pricking depth (that is, the length of the pricking needle protruding from the opening end 303 of the lancet case). Thus, the “pricking depth” can be adjusted by appropriately changing the “set position of the claw portion 137 in the lancet body 130” and/or the “set position of the stepped portion 357 in the lancet case 300”. As can be seen from the embodiments shown in FIG. 19, the claw portion 137 for defining the pricking depth, which is provided in the lancet body, preferably has flexibility to some degree so that the “ruffling of pricking needle” generated by the collision between the claw portion 137 and the stepped portion 357 is reduced. When the claw portion 137 of the lancet body collides with the stepped portion 357 of the lancet case, the vibration of the ruffling pricking needle can be generally caused by the collision (that is, the pathway for the pricking needle can be generally deflected). In this regard, the flexible claw portion 137, which is indirectly or directly connected to the pricking needle, can effectively absorb the ruffling energy. The claw portion 137 and stepped portion 357 may be formed of any kind of resin materials that can be used for lancets in general.

In some cases, the tip side of the lancet might be warped obliquely upward (i.e., in the direction of “P” shown in FIG. 19(c)) due to the collision between the claw portion 137 of the lancet body and the stepped portion 357 of the lancet case. In order to effectively prevent such “upwardly warped state” or “inclining-state” of the lancet, the above elastic portion 138 may be preferably provided at the upper side of the lancet body, that is, at the “the body side provided with the claw portion 137” as shown in FIG. 19(c).

### (Re-Use Preventing Mechanism)

The lancet pricking device 600 of the present invention preferably has a re-use preventing mechanism. As described above, after the first trigger part is displaced upon the pricking operation, the displaced state of the first trigger displaced is finally kept. That is, the displaced first trigger part cannot be returned to its original state of the pre-displacement. Specifically, as shown in FIG. 6, when the second trigger part 200B is pushed inwardly, the front end 210A of the first trigger part 200A moves inwardly, whereas the rear end 220A of the first trigger part 200A moves outwardly. As a result, the engagement part “a” (225A) of the first trigger part 200A comes into engagement with the engaged part “a” (240B) of the second trigger part 200B. Similarly, when the second trigger part 200B is pushed inwardly, the inward movement of the front end 210A of the first trigger part 200A causes the engagement between the engagement part “b” (255A) of the first trigger part 200A and the engaged part “b” (355) of the trigger support 350.

As can be seen from FIG. 6, even if the lancet body 130 is intended to go backward so as to obtain the compressed state of the spring again after the pricking, the displaced state of the first trigger part is secured, and thus the lancet body 130 cannot engage any longer with the first trigger part 200A (namely, the first trigger part cannot be returned to its original state of pre-launching). As a result, the used pricking device cannot any longer restore the original state of the pre-pricking, whereby the re-use of the pricking device is prevented.

### Embodiment of Use of Pricking Device

In the following, the embodiment of use of the lancet pricking device according to the present invention will be described. FIGS. 20(a) to 20(e) show the change of the lancet pricking device 600 over the time in numerical order.

The lancet pricking device 600 of the present invention before the pricking operation is shown in FIG. 20(a). First, as shown in FIGS. 20(a) and 20(b), the lancet cap 170 is removed from the lancet 100. The lancet cap 170 is removed preferably by twisting the lancet cap 170. Specifically, the lancet cap 170 (particularly, the holding portion 172) is rotated so that the “contact portion between the lancet body 130 and the lancet cap 170” is broken, and thereafter the lancet cap 170 is pulled forwardly as shown in FIG. 20(b). In other words, one hand holds the lancet pricking device from the outside, and the fingers of the other hand pull the lancet cap 170 while twisting the holder 172. As a result, the pricking needle 150 is exposed in the lancet body 130 within the lancet case 300. Then, after the opening end 303 of the lancet case 300 is applied to the predetermined region (e.g., finger) to be pricked of the patient's body (see FIG. 20(c)), the second trigger part 200B is pushed toward the inside of the lancet case (see FIG. 20(d)). The pushing of the second trigger part 200B causes the displacement of the first trigger part 200A, and thereby causing the ceasing of the engagement between the first trigger part 200A and the lancet body 130. Specifically, when the second trigger part 200B is pushed inwardly, the first trigger part 200A is pressed by the pushed second trigger part 200B wherein the front end 210A of the first trigger part 200A to move inwardly, whereas the rear end 220A of the first trigger part 200A moves outwardly. As a result, the locking of the lancet body 130 with respect to the first trigger part 200A is released. When the locking state is released (i.e., the engagement between the first trigger part 200A and the lancet body 130 ceases), the compressed spring 400 begins to expand, making it possible to launch the “lancet body 130 with the pricking needle 150 exposed” in the pricking direction.

The launched “lancet body 130 with the pricking needle 150 exposed” moves in the pricking direction while being guided along the inner wall surface of the lancet case. At this time, the elastic portion 138 of the moving lancet body slides on the inner wall surfaces 320b of the lancet case. Especially, the sliding elastic portion 138 preferably becomes warped and deformed such that its hollow portion becomes small and thin by the force received by the elastic portion 138 from the inner wall surface 320b of the lancet case. Such warped elastic portion 138 enables it to absorb the shock received in the lancet body 130 or the pricking needle 150 at the tine of pricking. As a result, the pricking needle 150 can moved more stably.

The launched lancet body 130 moves in the pricking direction, and thus the pricking needle 150 protrudes from the opening end 303 of the lancet case to prick the predetermined region (e.g., finger) of the body. The claw portion 137 of the moving lancet body makes contact with or hit the stepped portion 357 of the lancet case (see FIG. 19), and thereby the protruding length of the pricking needle from the opening end of the lancet case is restricted upon the pricking. Since, after the pricking, the expanded spring 400 is returned to its original shape, and thereby the pricking needle 150 is quickly retracted (see FIG. 20(e)). Specifically, since the “lancet body 130 with the pricking needle 150 exposed” has the spring 300 attached thereto, the lancet body 130 goes back such that it is pulled by the spring, and finally the pricking needle 150 is retracted into the lancet case 300. After the pricking operation, the displaced state of the first trigger part 200A is secured so that the lancet body 130 cannot any longer engage with the first trigger part 200A, making it impossible to return the device to the pre-launching stage.

It should be noted that the present invention as described above includes the following aspects:

The first aspect: A lancet pricking device comprising a lancet provided with a pricking needle, and a trigger for launching the pricking needle;

wherein the trigger comprises a first trigger part and a second trigger part disposed such as to cover the first trigger part;

the lancet is in engagement with the first trigger part at a point in time before the pricking needle is launched; and

when the second trigger part is pushed from the outside for the launching of the pricking needle, the displacement of the first trigger part is caused by the second trigger part, and thereby ceasing the engagement between the lancet and the first trigger part to launch the pricking needle.

The second aspect: The lancet pricking device according to the first aspect, wherein, a front end and a rear end of the first trigger part move in the reverse direction to each other in the displacement of the first trigger part.

The third aspect: The lancet pricking device according to the first or second aspect, further comprising a lancet case for housing the lancet, both of the first trigger part and the second trigger part are integrally formed with the lancet case.

The fourth aspect: The lancet pricking device according to any one of the first to third aspects, wherein each of the first and second trigger parts has an elongated shape; and

the elongated first and second trigger parts are superimposed over each other.

The fifth aspect: The lancet pricking device according to the fourth aspect, wherein the first trigger part has a pivot point at an inner side thereof, such that both of a front end and a rear end of the first trigger part serve as free ends; and

wherein the second trigger part has a rear end thereof serving as a fixed end and a front end thereof serving as a free end.

The sixth aspect: The lancet pricking device according to the fourth or fifth aspect, wherein, in the displacement of the first trigger part, the front end of the first trigger part is pressed inwardly due to contact of the first trigger part with the second trigger part, such that the front end of the first trigger part moves inwardly while the rear end of the first trigger part moves outwardly.

The seventh aspect: The lancet pricking device according to the sixth aspect, wherein the first trigger part has, at its rear end, an engagement part “a” for securing the trigger, whereas the second trigger part has, at its inner side, an engaged part “a” for securing the trigger; and

wherein, upon the displacement of the first trigger part, the engagement part “a” of the first trigger part engages with the engaged part “a” of the second trigger part, and thereafter the first trigger part cannot be returned to its original state of the pre-displacement thereof.

The eighth aspect: The lancet pricking device according to the sixth or seventh aspect, further comprising a trigger support connected to the first trigger part;

wherein the first trigger part has, at its front end, an engagement part “b” for securing the trigger, and the trigger support comprises an engaged part “b” for securing the trigger; and

wherein, upon the displacement of the first trigger part, the engagement part “b” of the first trigger part engages with the engaged part “b” of the trigger support, and thereafter the first trigger part cannot be returned to its original state of the pre-displacement thereof.

The ninth aspect: The lancet pricking device according to the seventh or eighth aspect, wherein, due to the effect that the first trigger part cannot be returned to its original state of the pre-displacement thereof, the lancet cannot any longer engage with the first trigger part so that the device cannot any longer restore its original state after the launching of the pricking needle.

The tenth aspect: The lancet pricking device according to the eighth aspect, wherein the trigger support is integrally formed with the lancet case.

The eleventh aspect: The lancet pricking device according to any one of the eighth to tenth aspects, wherein a lancet cap for covering the pricking needle is provided with a protrusion; and

wherein, while the pricking needle is covered with the lancet cap, the protrusion of the lancet cap is positioned directly under the second trigger part such as to be able to abut against the second trigger part, and thereby the displacement of the first trigger part is prevented even if the second trigger part is pushed from the outside.

The twelfth aspect: The lancet pricking device according to the eleventh aspect when appendant to the eighth aspect, wherein the protrusion of the lancet cap is positioned between the trigger support and the front end of the second trigger part.

The thirteenth aspect: The lancet pricking device according to the eleventh or twelfth aspect, wherein, after the lancet cap is removed from the pricking needle, the second trigger part can be pushed inwardly by pressing it from the outside, and thereby causing the displacement of the first trigger part.

The fourteenth aspect: A lancet pricking device comprising a lancet, a launching spring and a lancet case that houses the lancet and the spring, wherein

the lancet comprises a lancet body, a lancet cap and a pricking needle in which the pricking needle is situated in both of the lancet body and the lancet cap;

a trigger integrally provided with the lancet case comprises a first trigger part and a second trigger part positioned such as to cover the first trigger part;

wherein the spring is attached to the lancet body, and the lancet body is in engagement with the first trigger part such that the spring is kept compressed before the pricking needle is launched; and

wherein, after the lancet cap is removed, the second trigger part can be pushed inwardly by pressing it from the outside to displace the first trigger part, and thereby ceasing the engagement between the lancet body and the first trigger part.

The fifteenth aspect: The lancet pricking device according to the fourteenth aspect, further comprising an exterior component that encloses at least a part of the lancet case.

The sixteenth aspect: The lancet pricking device according to the fourteenth or fifteenth aspect, wherein the launching spring is attached to a spring-attachment portion provided at a rear-sided inner wall of the lancet case; and

wherein the spring-attachment portion has a bent form of an elongated part with a free end and a fixed end.

The seventeenth aspect: A lancet pricking device comprising a lancet case that houses the lancet:

wherein a launching spring is attached to a spring-attachment portion provided at a rear-sided inner wall of the lancet case; and

the spring-attachment portion has a bent form of an elongated part with a free end and a fixed end.

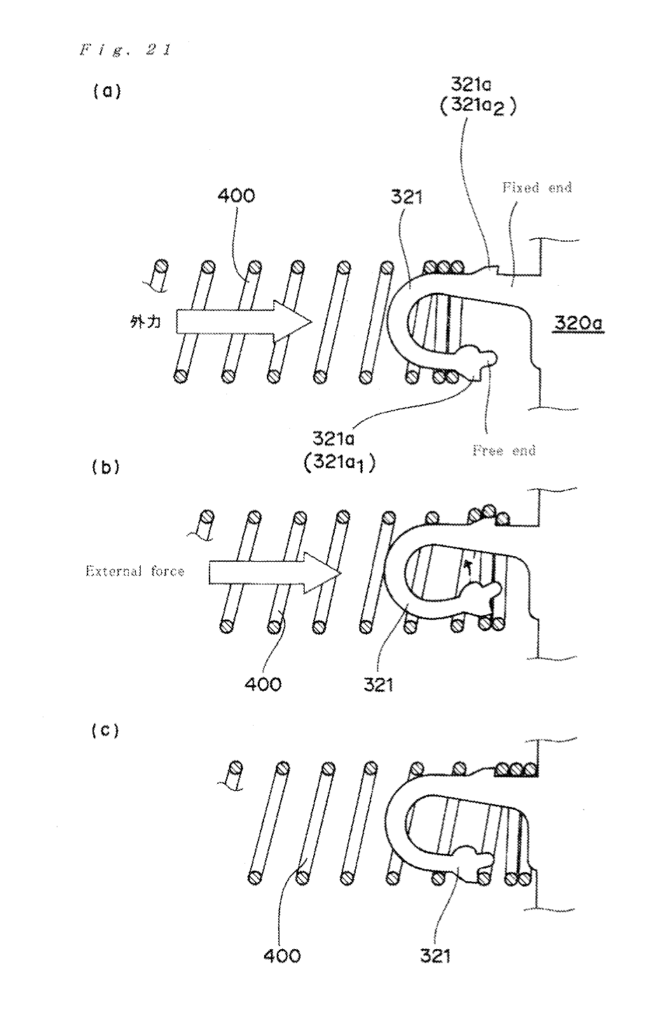

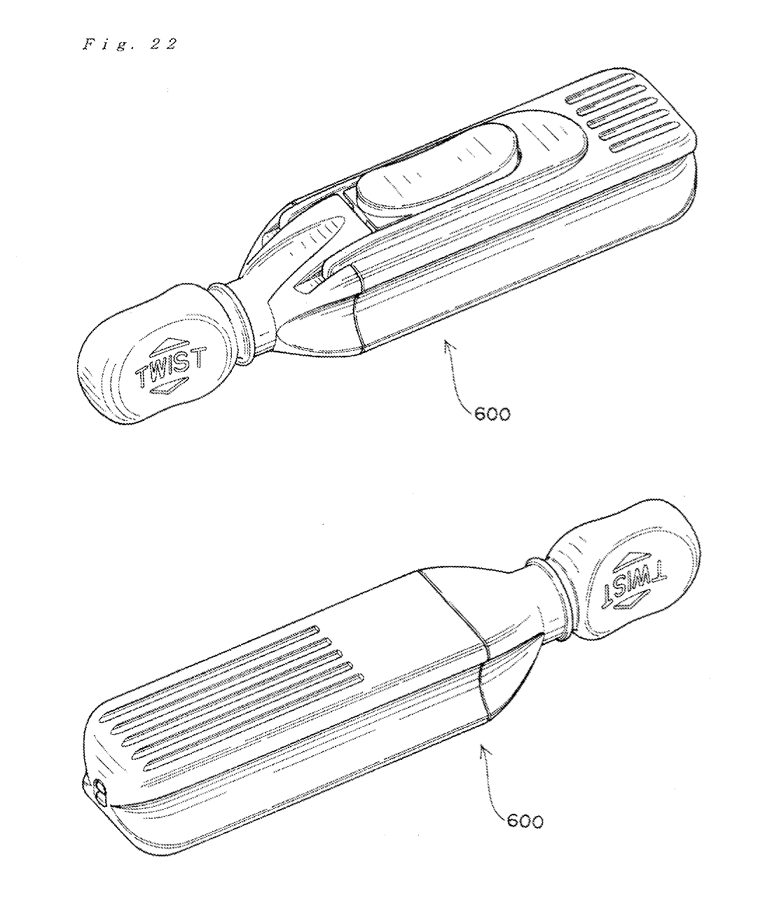

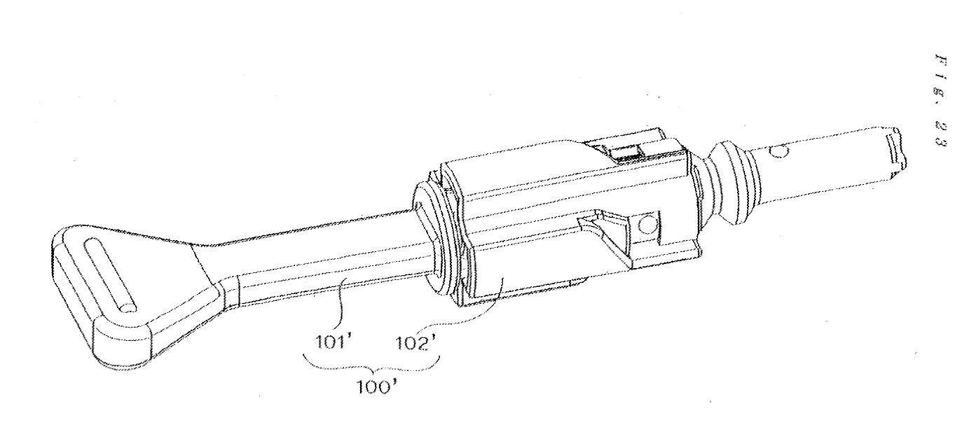

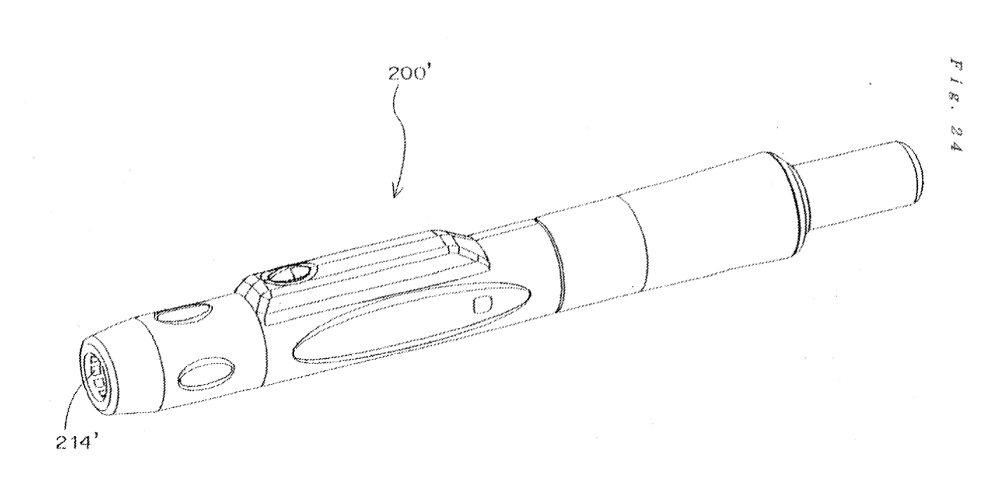

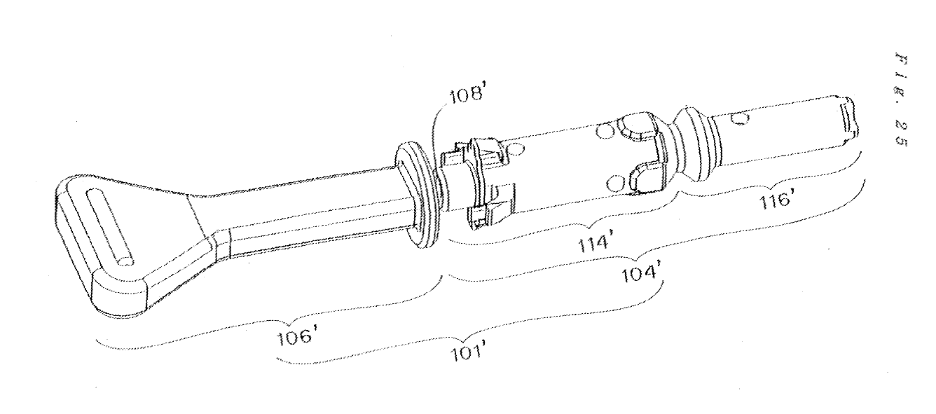

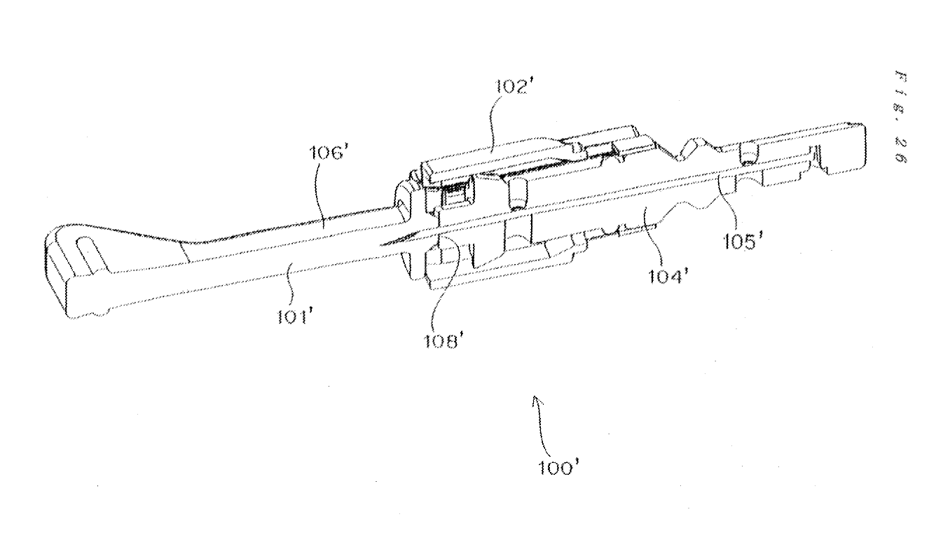

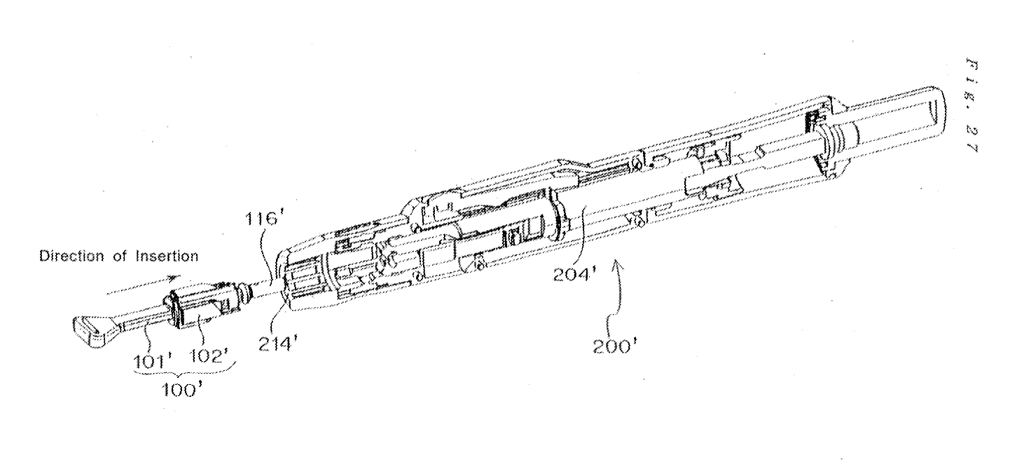

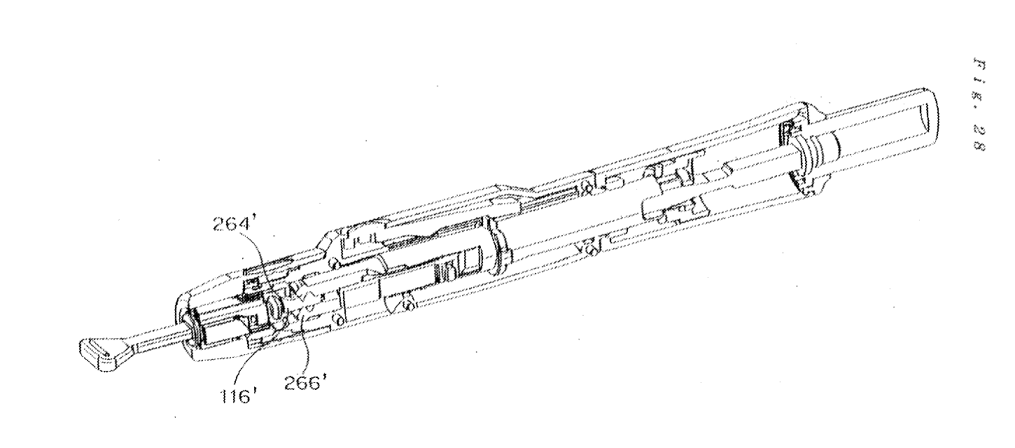

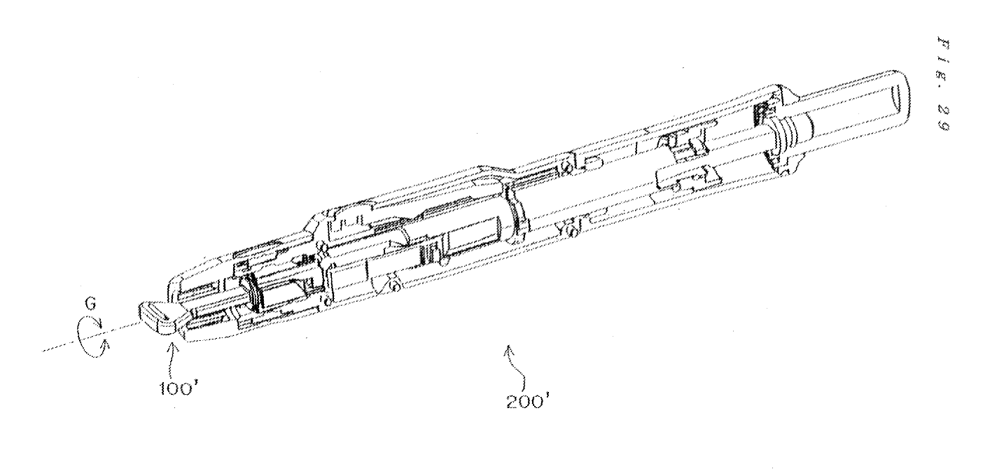

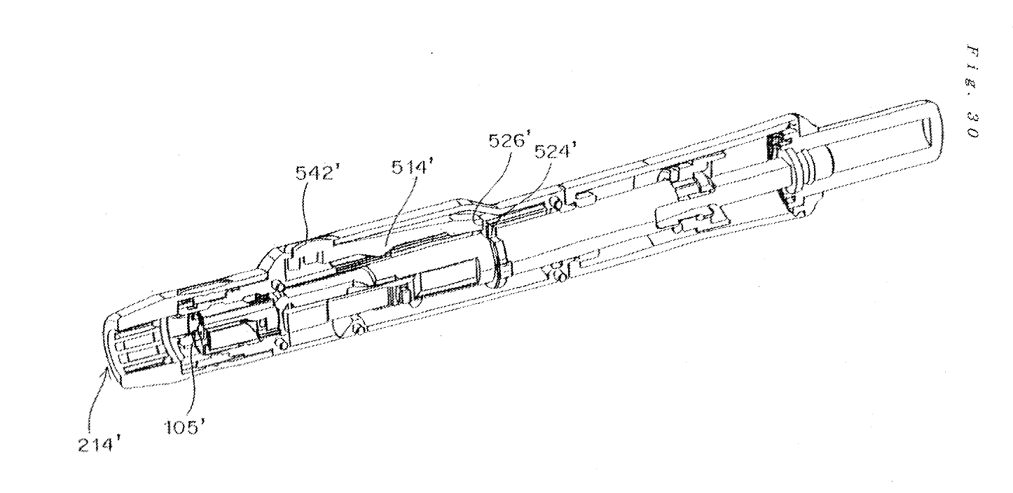

Although a few embodiments of the present invention have been hereinbefore described, the present invention is not limited to these embodiments. It will be readily appreciated by those skilled in the art that various modifications are possible without departing from the scope of the present invention. For example, the following modified embodiments are possible.
The trigger mechanism of the present invention, which has been described, is composed of the two elements, namely, the first trigger part and the second trigger part. However, the trigger mechanism is not necessarily limited thereto, and may be composed of three or more elements. For example, the displacement of the first trigger part may be caused by the pressing of the second trigger part, and then the displacement of the third trigger part may be further caused by the displacement of the first trigger part, and thereby ceasing the engagement of the lancet with respect to the third trigger part.
The elastic portion with a hollow structure has been described. However, the elastic portion is not necessarily limited thereto. The elastic portion may be made of the elastic material as a whole. That is, the elastic portion may be made of material with no hollow structure, the material enabling an elastic deformation thereof. In this regard, the elastic portion is made of rubber or elastomer material, for example.
As for the improved straight pricking pathway of the needle regarding the elastic portion, the moving of the needle in the pricking direction has been mainly described. However, the present invention is not necessarily limited to this. According to the present invention, the straight pricking pathway of the needle moving backwardly upon the pricking operation is also improved in the same manner. That is, after the pricking, the backward-moving elastic portion slides on the inner wall surface of the lancet case, and thereby the shock received in the pricking needle can be effectively absorbed by the elastic portion, making it possible to stabilize the backward movement of the pricking needle. This means that the pathway of the pricking needle moving backwardly is linear, like the pathway of the pricking, needle moving forwardly. In particular, the pricking needle backwardly moves more linearly immediately after the pricking, so that the “phenomenon of adversely hollowing the pricked portion by the backward moving needle” is effectively reduced, which can effectively reduce the pain of the pricked subject. Further, the lancet body's elastic portion sliding on the inner wall of the lancet case has been described. However, the present invention is not necessarily limited thereto. According to the present invention, the elastic portion may be provided not in the lancet body, but also at the inner wall of the lancet case.
The lancet pricking device of the present invention is not limited to the appearance as shown in FIG. 10. Various kinds of appearances are possible. For example, the lancet pricking device of the present invention may have the appearance as shown in FIG. 22.

Finally, as an additional remark, the “spring attachment portion having the form of a hook” will be described below. The hook form, i.e., the bent form of an elongated part with a free end and a fixed end can serve to easily perform an assembly of the lancet pricking device. During the assembly of the device, the launching spring is introduced from the front opening end of the lancet case (introduced from the opening end as deeply as possible), and then the lancet is inserted into the lancet case from the opening end thereof till the lancet reached the rear end of the case. With the insertion of the lancet, the launching spring becomes compressed, within the case, between the hook-like attachment portion and the rear end-attachment portion of the lancet. For example, the free length of the launching spring of 17 mm is compressed by about 7 mm into the length of 10 mm. A spring compression force at this compressed state of the spring is, for example, about 3 N. It is thus necessary that the spring engages with (fitted into) the spring-attachment portion and the lancet by such force. Further, the engagement is required to prevent a releasing of the spring after the pricing operation (in a case where the spring is extended from the free length of 17 mm up to about 20 mm, the tensile force of the spring becomes about 1 N, and thus it is necessary that the ceasing of the engagement is prevented even under such a force). Taking into consideration these circumstances, the launching spring has the strength enough not to be deformed (of the both end-contact type). An object to be engaged with the spring is required to be easily warped, to be less likely to be released, and to be capable of being manufactured. In order to put such spring in practical use, the form of hook of the spring-attachment portion of the lancet case is employed. The lancet case is generally made of materials (e.g., POM, PS, ABS or the like) harder than the material (LDPE) of the lancet, and thus the attachment portion of the lancet case employs the form of fishing needle capable of being easily warped.

### INDUSTRIAL APPLICABILITY

The lancet pricking device according to the present invention is prevented from being re-used, and also has an improved pricking pathway of the needle thereof. Accordingly, the lance pricking device of the present invention can be not only used to take out blood from a patient with diabetes, but also suitably used for various other applications that need the blood sampling.

### CROSS REFERENCE TO RELATED PATENT APPLICATION

The present application claims the right of priority of Japan patent application No. 2010-232659 (filing date: Oct. 15, 2010, title of the invention: LANCET PRICKING DEVICE), the whole contents of which are incorporated herein by reference.

### BRIEF EXPLANATION OF REFERENCE NUMERALS

100 Lancet
130 Lancet body
133 Rear end of lancet (spring-attachment portion)
135 Engaging portion (locking portion) of lancet body
137 Claw portion for defining or adjusting pricking depth
138 Elastic portion
138a Cavity or hollow of elastic portion
145 Hollow portion of lancet
150 Pricking needle
150a Tip of pricking needle
170 Lancet cap
172 Holding portion of lancet cap
175 Protrusion of lancet cap
200 Trigger
200A First trigger part
210A Front end of first trigger part
212A Inner side (lower surface) of front end of first trigger part
215A Front side of first trigger part
220A Rear end of first trigger part
225A Trigger-securing engagement part “a” of first trigger part
230A Engaged portion of first trigger part
250A Pivot point of first trigger part
255A Trigger-securing engagement part “b” of first trigger part
200B Second trigger part
201B Inner side (lower surface) of second trigger part
210B Front end of second trigger part
220B Rear end of second trigger part
240B Trigger-securing engagement part “a” of second trigger part
300 Lancet case
303 Opening end of lancet case
320a Rear end sided-inner wall surface of lancet case
320b Inner wall surface of lancet case
321 Spring-attachment portion provided inside lancet case
321a Protrusion of spring-attachment portion
321a₁ First protrusion of spring-attachment portion
321a₂ Second protrusion of spring-attachment portion
350 Trigger support (nearly equal to a coupled portion of first trigger part in case)
355 Trigger-securing engaged part “b” of trigger support
357 Stepped portion for defining or adjusting pricking depth
390 Space formed under second trigger part
400 launching spring
500 Exterior component
550 Cutout of exterior component
600 Lancet pricking device
100′ Lancet assembly
101′ Lancet
102′ Protective cover
104′ Lancet body
105′ Pricking needle
106′ Lancet cap
108′ Weakened part
114′ Front part of lancet body
116′ Rear part of lancet body
200′ Injector
204′ Plunger
214′ Front end opening of injector
264′, 266′ Tips of plunger
514′ Trigger component
524′ Protrusion provided in plunger
526′ Rear end portion of trigger component
542′ Press portion of trigger component

1. A lancet pricking device comprising a lancet equipped with a pricking needle, and a trigger for launching the pricking needle;
wherein the trigger comprises a first trigger part and a second trigger part disposed such as to cover the first trigger part;
the lancet is in engagement with the first trigger part before the pricking needle is launched; and
when the second trigger part is pushed for the launching of the pricking needle, the first trigger part is displaced by the second trigger part, and thereby ceasing the engagement of the lancet with the first trigger part to launch the pricking needle.

2. The lancet pricking device according to claim 1, wherein, a front end and a rear end of the first trigger part move in the reverse direction to each other in the displacement of the first trigger part.

3. The lancet pricking device according to claim 1, further comprising a lancet case for housing the lancet, both of the first trigger part and the second trigger part are integrally formed with the lancet case.

4. The lancet pricking device according to claim 1, wherein each of the first and second trigger parts has an elongated shape; and
the elongated first and second trigger parts are arranged to be superimposed over each other.

5. The lancet pricking device according to claim 4, wherein the first trigger part has a pivot point at an inner side thereof, such that both of a front end and a rear end of the first trigger part serve as free ends; and
wherein the second trigger part has a rear end thereof serving as a fixed end and a front end thereof serving as a free end.

6. The lancet pricking device according to claim 4, wherein, in the displacement of the first trigger part, the front end of the first trigger part is pressed inwardly due to contact of the first trigger part with the second trigger part, such that the front end of the first trigger part moves inwardly while the rear end of the first trigger part moves outwardly.

7. The lancet pricking device according to claim 6, wherein the first trigger part has, at its rear end, an engagement part “a” for securing the trigger, whereas the second trigger part has, at its inner side, an engaged part “a” for securing the trigger; and
wherein, upon the displacement of the first trigger part, the engagement part “a” of the first trigger part engages with the engaged part “a” of the second trigger part, and thereafter the first trigger part cannot be returned to its original state of the pre-displacement thereof.

8. The lancet pricking device according to claim 6, further comprising a trigger support connected to the first trigger part;
wherein the first trigger part has, at its front end, an engagement part “b” for securing the trigger, and the trigger support comprises an engaged part “b” for securing the trigger; and
wherein, upon the displacement of the first trigger part, the engagement part “b” of the first trigger part engages with the engaged part “b” of the trigger support, and thereafter the first trigger part cannot be returned to its original state of the pre-displacement thereof.

9. The lancet pricking device according to claim 7, wherein, due to the effect that the first trigger part cannot be returned to its original state of the pre-displacement thereof, the lancet cannot any longer engage with the first trigger part so that the device cannot any longer restore its original state after the launching of the pricking needle.

10. The lancet pricking device according to claim 8, wherein the trigger support is integrally formed with the lancet case.

11. The lancet pricking device according to claim 8, wherein a lancet cap for covering the pricking needle is provided with a protrusion; and
wherein, while the pricking needle is covered with the lancet cap, the protrusion of the lancet cap is positioned directly under the second trigger part such as to be able to abut against the second trigger part, and thereby the displacement of the first trigger part is prevented even if the second trigger part is pushed from the outside.

12. The lancet pricking device according to claim 8, wherein a lancet cap for covering the pricking needle is provided with a protrusion;
wherein, while the pricking needle is covered with the lancet cap, the protrusion of the lancet cap is positioned directly under the second trigger part such as to be able to abut against the second trigger part, and thereby the displacement of the first trigger part is prevented even if the second trigger part is pressed from the outside; and
wherein the protrusion of the lancet cap is positioned between the trigger support and the front end of the second trigger part.

13. The lancet pricking device according to claim 11, wherein, after the lancet cap is removed from the pricking needle, the second trigger part can be pushed inwardly by pressing it from the outside, and thereby causing the displacement of the first trigger part.

14. A lancet pricking device comprising a lancet, a launching spring and a lancet case that houses the lancet and the spring, wherein
the lancet comprises a lancet body, a lancet cap and a pricking needle in which the pricking needle is situated in both of the lancet body and the lancet cap;
a trigger integrally provided with the lancet case comprises a first trigger part and a second trigger part positioned such as to cover the first trigger part;
wherein the spring is attached to the lancet body, and the lancet body is in engagement with the first trigger part such that the spring is kept compressed before the pricking needle is launched; and
wherein, after the lancet cap is removed, the second trigger part can be pushed inwardly by pressing it from the outside to displace the first trigger part, and thereby ceasing the engagement of the lancet body with the first trigger part.

15. The lancet pricking device according to claim 14, further comprising an exterior component that encloses at least a part of the lancet case.

16. The lancet pricking device according to claim 14, wherein the launching spring is attached to a spring-attachment portion provided at a rear-sided inner wall of the lancet case; and
wherein the spring-attachment portion has a bent form of an elongated part with a free end and a fixed end.

17. A lancet pricking device comprising a lancet case that houses the lancet:
wherein a launching spring is attached to a spring-attachment portion provided at a rear-sided inner wall of the lancet case; and
the spring-attachment portion has a bent form of an elongated part with a free end and a fixed end.
